(19) United States
(12) Patent Application Publication
(10) Pub. No.: US 2012/0244122 A1
(43) Pub. Date: Sep. 27, 2012

(54) Peptides for the Treatment of HCV Infections

(76) Inventor: Craig E. Masse, Cambridge, MA (US)

(21) Appl. No.: 13/321,785

(22) PCT Filed: May 28, 2010
(86) PCT No.: PCT/US10/36682

§ 371 Date: Jun. 6, 2012

(30) Foreign Application Priority Data
May 28, 2009 (US) 61217185

(51) Int. Cl. A61K 38/06; A61K 38/21; A61P 31/14; C07K 5/087

(52) U.S. Cl. 424/85.7; 530/331; 514/21.9; 514/4.3

(57) ABSTRACT

This invention relates to novel compounds that are peptides derivatives and pharmaceutically acceptable salts thereof. More specifically, this invention relates to novel peptides that are deuterated derivatives of boceprevir. This invention also provides compositions comprising one or more compounds of this invention and a carrier and the use of the disclosed compounds and compositions in methods of treating HCV infection.

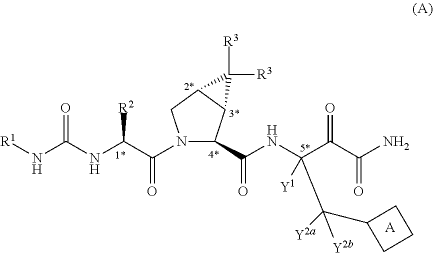

### RELATED APPLICATION

This application claims the benefit of U.S. Provisional Patent Application No. 61/217,185, filed May 28, 2009. The entire teachings of the above application is incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug. Boceprevir, also known as SCH-503034, or as N-(4-amino-1-cyclobutyl-3,4-dioxobutan-2-yl)-3-(2-(3-tert-butylureido)-3,3-dimethylbutanoyl)-6,6-dimethyl-3-azabicyclo[3.1.0]hexane-2-carboxamide, is a promising drug candidate for the treatment of hepatitis C virus (HCV). HCV is a (+)-sense single-stranded RNA virus that has been implicated as the major causative agent in non-A, non-B hepatitis (NANBH). Boceprevir acts by inhibiting HCV NS3/NS4a serine protease. Of the several non-structural proteins (NS1, NS2, NS3, NS4a, NS5a, and NS5b) contained in the HCV protease, HCV NS3 serine protease is responsible for proteolysis of the polypeptide at the NS3/NS4a, NS4a/NS4b, NS4b/NS5a, and NS5a/NS5b junctions and is thus responsible for generating five viral proteins during viral replication. The NS4a protein is a co-factor for the serine protease activity of NS3. (See International Patent Publication no. WO 2002008244).

Boceprevir is currently in phase III clinical trials for treatment of hepatitis C. In a phase II clinical trial, high sustained viral response (SVR) rates (66-74% after 12 weeks, 55-56% after 24 weeks) were achieved in treatment-naïve patients dosed with boceprevir combined with peginterferon alpha-2b and ribavirin (DailyDrugNews.com (Daily Essentials) Aug. 5, 2008; Kwo, P et al., 43rd Annu Meet Eur Assoc Study Liver (EASL) (April 23-27, Milan) 2008, Abst). SVR rates in non-responders (patients who did not respond to or who experienced rebound infection with PEG-interferon+ribavirin) were significantly lower than for treatment-naïve patients (Schiff, E et al., EASL 2008, Abstract 104, p. 34).

Although boceprevir has been shown to have high potency (similar to telaprevir, which is another leading HCV protease inhibitor), its overall reduction in viral load is lower than its high potency would predict. This discrepancy has been attributed to inadequate exposure of the drug in humans. Studies of boceprevir as monotherapy in non-responders have shown that a clear relationship exists between Cₘᵢₙ and viral load reduction (Zeuzem, S et al., 56th Annu Meet Am Assoc Study Liver Dis, 2005, (November 11-15, San Francisco): Abst 201; Kempf, D J et al., Antivir. Chem. and Chemotherapy, 2007, 18(3): 163-67) indicating that increasing the Cₘᵢₙ of boceprevir could enhance efficacy.

In general, boceprevir is well-tolerated in patients with hepatitis C. Adverse events were infrequent and included headache, fever and myalgia. (Sarrazin, C et al., Gastroenterology, 2007, 132(4): 1270 and Zeuzem, S et al., 56th Annu Meet Am Assoc Study Liver Dis, 2005, (November 11-15, San Francisco): Abst 201).

Despite the beneficial activities of boceprevir, there is a continuing need for new compounds to treat hepatitis C.

### SUMMARY OF THE INVENTION

This invention relates to novel compounds that are peptide derivatives and pharmaceutically acceptable salts thereof. More specifically, this invention relates to novel peptides that are derivatives of boceprevir. This invention also provides pharmaceutical compositions comprising one or more compounds of this invention and a pharmaceutically acceptable carrier and the use of the disclosed compounds and compositions in methods of treating diseases and conditions that are beneficially treated by administering an HCV NS3/NS4A protease inhibitor, such as boceprevir.

### BRIEF DESCRIPTION OF THE DRAWINGS

FIG. 1 depicts the comparative stability of compounds of this invention and other derivatives of boceprevir as compared to boceprevir in human liver microsomes.

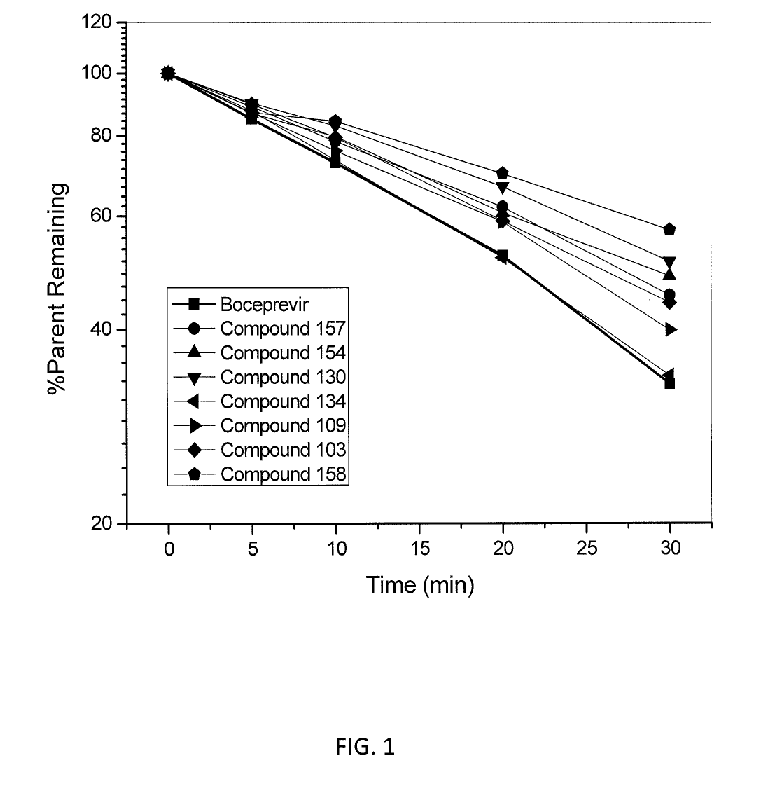

### DETAILED DESCRIPTION OF THE INVENTION

The terms “ameliorate” and “treat” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of boceprevir will inherently contain small amounts of deuterated isotopologues. The concentration of naturally abundant stable hydrogen isotopes, notwithstanding this variation, is small and immaterial as compared to the degree of stable isotopic substitution of compounds of this invention. See, for instance, Wada, E et al., Seikagaku, 1994, 66: 15; Gannes, L Z et al., Comp Biochem Physiol Mol Integr Physiol, 1998, 119: 725.

The compounds of the present invention are distinguished from such naturally occurring minor forms in that the term “compound” as used in this invention refers to a composition of matter that has a minimum isotopic enrichment factor at least 3000 (45% deuterium incorporation) for each deuterium atom that is present at a site designated as a site of deuteration in Formula (I).

In the compounds of the invention, any atom not specifically designated as a particular isotope is meant to represent any stable isotope of that atom unless otherwise stated. Unless otherwise stated, when a position is designated specifically as “H” or “hydrogen,” the position is understood to have hydrogen at its natural abundance isotopic composition. Also unless otherwise stated, when a position is designated specifically as “D” or “deuterium”, the position is understood to have deuterium at an abundance at least 3000 times the natural abundance of deuterium, which is 0.015% (i.e., at least 45% deuterium incorporation).

The term “isotopic enrichment factor” as used herein means the ratio between the isotopic abundance and the natural abundance of a specified isotope.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The structural formula depicted herein may or may not indicate whether atoms at certain positions are isotopically enriched. In a most general embodiment, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, the stable isotopes are present at natural abundance at all positions in a compound not specifically designated as being isotopically enriched.

The term “isotopologue” refers to a species in which the chemical structure differs from a specific compound of this invention only in the isotopic composition thereof. Isotopologues can differ in the level of isotopic enrichment at one or more positions and/or in the positions(s) of isotopic enrichment.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 55% of the compound. In other embodiments, the relative amount of such isotopologues in toto will be less than 50%, less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

Compounds of Formula A have multiple chiral centers, at least in the positions indicated below with an *. The configuration of chiral center 1 depicted in Formula A (1*) is “S”, the configuration of chiral center 2 depicted in Formula A (2*) is “S”, the configuration of chiral center 3 depicted in Formula A (3*) is “R”, and the configuration of chiral center 4 depicted in Formula A (4*) is “S.” The configuration at chiral center 5 (5*) is not specified in Formula A and includes the “S” configuration, the “R” configuration and a mixture of the “S” and “R” configurations.

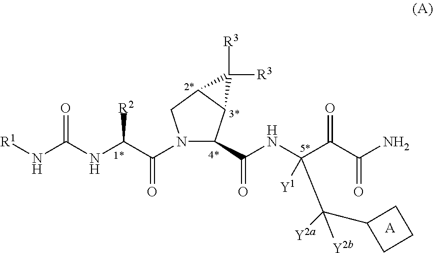

In one embodiment, the configuration at each of the chiral centers 1-4 is at least 90% of that depicted in Formula A and the configuration at chiral center 5 ranges from 0-100% “S.” In a more particular embodiment, the configuration at each of the chiral centers 1-4 is at least 92% of that depicted in Formula A and the configuration at chiral center 5 ranges from 0-100% “S.” In an even more particular embodiment, the configuration at each of the chiral centers 1-4 is at least 95% of that depicted in Formula A and the configuration at chiral center 5 ranges from 0-100% “S.”

In another embodiment, the configuration at each of the chiral centers 1-4 is at least 90% of that depicted in Formula A and the configuration at chiral center 5 ranges from 50-100% “S” (e.g., from about 60-80% “S”). In a more particular embodiment, the configuration at each of the chiral centers 1-4 is at least 92% of that depicted in Formula A and the configuration at chiral center 5 ranges from 50-100% “S” (e.g., from about 60-80% “S”). In an even more particular embodiment, the configuration at each of the chiral centers 1-4 is at least 95% of that depicted in Formula A and the configuration at chiral center 5 ranges from 50-100% “S” (e.g., from about 60-80% “S”).

In another embodiment, the configuration at each of the chiral centers 1-4 is at least 90% of that depicted in Formula A and the configuration at chiral center 5 ranges from 0-50% “S” (e.g., from about 20-40% “S”). In a more particular embodiment, the configuration at each of the chiral centers 1-4 is at least 92% of that depicted in Formula A and the configuration at chiral center 5 ranges from 0-50% “S” (e.g., from about 20-40% “S”). In an even more particular embodiment, the configuration at each of the chiral centers 1-4 is at least 95% of that depicted in Formula A and the configuration at chiral center 5 ranges from 0-50% “S” (e.g., from about 20-40% “S”).

From 0-100% “S” includes any value from 0 to 100, for example, 1, 2, 3, 4, 5, etc. In addition, from 0-100% “S” includes defined ranges, such as from about 0-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95% and 95-100%, as well as broader ranges, such as from about 0-20%, 20-40%, 40-60%, 60-80% and 80-100%.

From 50-100% “S” includes any value from about 50 to 100, for example, 51, 52, 53, 54, 55 etc. In addition, from 50-100% “S” includes defined ranges, such as from about 50-55%, 55-60%, 60-65%, 65-70%, 70-75%, 75-80%, 80-85%, 85-90%, 90-95% and 95-100%, as well as broader ranges such as from about 50-90, for example, from about 60-80%.

From 0-50% “S” includes any value from about 0 to 50, for example, 1, 2, 3, 4, 5 etc. In addition, from 50-100% “S” includes defined ranges, such as from about 0-5%, 5-10%, 10-15%, 15-20%, 20-25%, 25-30%, 30-35%, 35-40%, 40-45% and 45-50%, as well as broader ranges such as from about 0-40, for example, from about 20-40%.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula A:

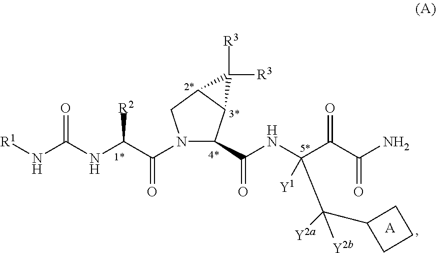

or a pharmaceutically acceptable salt thereof, wherein:

the configuration at each of chiral centers 1-4 is at least 90% of that depicted;

the configuration at carbon atom 5 is from 0-100% S;

Ring A is a cyclobutyl ring having 0-7 deuterium atoms;

each of R¹ and R² is independently —C(CH₃)₃, wherein 1 to 9 hydrogen atoms are optionally replaced with deuterium atoms;

each R³ is independently selected from —CH₃, —CH₂D, —CHD₂, and —CD₃; and

each Y is independently selected from hydrogen and deuterium;

provided that at least one Y² is deuterium when R¹ and R² are simultaneously —C(CH₃)₃, R³ is —CH₃, and ring A has zero deuterium atoms; and

further provided that when R¹ and R² are —C(CD₃)₃, Y¹ is hydrogen, each Y² is hydrogen, and Ring A has zero deuterium atoms, each R³ is not CD₃ or each R³ is not CH₃.

In one embodiment, R¹ is —C(CH₃)₃ or —C(CD₃)₃; R² is —C(CH₃)₃ or —C(CD₃)₃; each R³ group is the same; and each Y² is the same. In one aspect of this embodiment, R³ is selected from —CH₃ and —CD₃. In another aspect of this embodiment, R³ is —CD₃. In yet another aspect of this embodiment, Ring A is a cyclobutyl ring having either 0 deuterium atoms or 7 deuterium atoms. In still another aspect of this embodiment, Ring A is a cyclobutyl ring having either 0 deuterium atoms or 7 deuterium atoms and R³ is —CD₃. In one aspect of this embodiment each Y² is hydrogen. In another aspect of this embodiment, each Y² is deuterium.

Another embodiment of the invention provides a compound of Formula A wherein, Ring A is a cyclobutyl ring having either 0 deuterium atoms or 7 deuterium atoms. In one aspect of this embodiment, R¹ and R² are the same; each R³ is the same; and each Y is the same. In another aspect of this embodiment, R¹ and R² are the same and are selected from —C(CH₃)₃ and —C(CD₃)₃; and each R³ is the same and is selected from —CH₃ and —CD₃. In one aspect of this embodiment each Y² is hydrogen. In another aspect of this embodiment, each Y² is deuterium.

Other embodiments of Formula A include those wherein:

a) R¹ is selected from —C(CH₃)₃ and —C(CD₃)₃;

b) R² is selected from —C(CH₃)₃ and —C(CD₃)₃;

c) each R³ is the same;

d) each R³ is independently selected from —CH₃ and —CD₃;

e) Ring A is a cyclobutyl ring having 0 or 7 deuterium atoms; or

f) each Y² is the same.

In one aspect of embodiment c), each R³ is —CH₃. In another aspect of embodiment c), each R³ is —CD₃. In one aspect of embodiment f), each Y² is hydrogen. In another aspect of embodiment f), each Y² is deuterium.

In another embodiment, a compound of Formula A has the characteristics set forth in one or more of a) through f), above; and at least one of R¹ and R² is —C(CD₃)₃, or each R³ is —CD₃. For example, at least one of R¹ and R² is —C(CD₃)₃, or each R³ is —CD₃; and Ring A is a cyclobutyl ring having 0 or 7 deuterium atoms. In another embodiment, at least one of R¹ and R² is —C(CD₃)₃, or each R³ is —CD₃; and each Y² is the same. In one aspect of this embodiment each Y² is hydrogen. In another aspect of this embodiment, each Y² is deuterium.

In still another embodiment, a compound of Formula A has the characteristics set forth in two or more of a) through f), above. Such combinations include, but are not limited to: a and b; a and c; b and c; a, b and c; a and d; b and d; a, b and d; a, c and d; b, c, and d; a, b, c and d; e and c; e and d; e and a; e and b; e, b and a; e, c and a; e, c and b; e, c, b and a; e, d and a; e, d and b; e, d, b and a; e, d, c and a; e, d, c and b; e, d, c, b and a; a and f; b and f; a, b and f; c and f; a, c, and f; b, c and f; a, b, c and f; d and f; b and f; a, b, d and f; a, c, d and f; b, c, d and f; a, b, c, d, and f.

In still another embodiment, each of R¹ and R² is —C(CD₃)₃, each R³ is the same and is selected from —CD₃ and —CH₃, each Y² is the same, and Ring A is selected from a cyclobutyl ring having 0 deuterium atoms and a cyclobutyl ring having 7 deuterium atoms. In one aspect of this embodiment each Y² is hydrogen. In another aspect of this embodiment, each Y² is deuterium.

Examples of specific compounds of Formula A are shown in Table 1a below. In these examples, Y^{2a} is the same as Y^{2b} and Ring A has zero deuterium atoms (H₇) or seven deuterium atoms (D₇) replacing hydrogen atoms at available ring carbon positions.

TABLE 1a
Examples of Specific Compounds of Formula A

| Compound | R¹ | R² | each R³ | Y¹ | each Y² | Ring A |
|---|---|---|---|---|---|---|
| 100 | C(CD₃)₃ | C(CD₃)₃ | CD₃ | D | D | D₇ |
| 101 | C(CD₃)₃ | C(CD₃)₃ | CD₃ | D | D | H₇ |
| 102 | C(CD₃)₃ | C(CD₃)₃ | CD₃ | D | H | H₇ |
| 104 | C(CD₃)₃ | C(CD₃)₃ | CD₃ | H | H | D₇ |
| 105 | C(CD₃)₃ | C(CD₃)₃ | CD₃ | D | H | D₇ |
| 106 | C(CD₃)₃ | C(CD₃)₃ | CH₃ | D | D | D₇ |
| 107 | C(CD₃)₃ | C(CD₃)₃ | CH₃ | H | D | D₇ |
| 108 | C(CD₃)₃ | C(CD₃)₃ | CH₃ | H | H | D₇ |
| 110 | C(CD₃)₃ | C(CD₃)₃ | CH₃ | H | D | H₇ |
| 111 | C(CD₃)₃ | C(CD₃)₃ | CH₃ | D | D | H₇ |
| 112 | C(CD₃)₃ | C(CD₃)₃ | CH₃ | D | H | H₇ |
| 113 | C(CD₃)₃ | C(CH₃)₃ | CD₃ | D | D | D₇ |
| 114 | C(CD₃)₃ | C(CH₃)₃ | CD₃ | H | D | D₇ |
| 115 | C(CD₃)₃ | C(CH₃)₃ | CD₃ | H | H | D₇ |
| 116 | C(CD₃)₃ | C(CH₃)₃ | CD₃ | D | D | H₇ |
| 117 | C(CD₃)₃ | C(CH₃)₃ | CD₃ | H | D | H₇ |
| 118 | C(CD₃)₃ | C(CH₃)₃ | CD₃ | D | H | H₇ |
| 119 | C(CD₃)₃ | C(CD₃)₃ | CD₃ | H | H | H₇ |
| 120 | C(CH₃)₃ | C(CD₃)₃ | CD₃ | D | D | D₇ |
| 121 | C(CH₃)₃ | C(CD₃)₃ | CD₃ | H | D | D₇ |
| 122 | C(CH₃)₃ | C(CD₃)₃ | CD₃ | H | H | D₇ |
| 123 | C(CH₃)₃ | C(CD₃)₃ | CD₃ | D | H | D₇ |
| 124 | C(CH₃)₃ | C(CD₃)₃ | CD₃ | D | D | H₇ |
| 125 | C(CH₃)₃ | C(CD₃)₃ | CD₃ | H | D | H₇ |
| 126 | C(CH₃)₃ | C(CD₃)₃ | CD₃ | H | H | H₇ |
| 127 | C(CH₃)₃ | C(CD₃)₃ | CD₃ | D | H | H₇ |
| 128 | C(CH₃)₃ | C(CH₃)₃ | CD₃ | D | D | D₇ |
| 129 | C(CH₃)₃ | C(CH₃)₃ | CD₃ | D | H | D₇ |
| 130 | C(CH₃)₃ | C(CH₃)₃ | CD₃ | H | D | D₇ |
| 131 | C(CH₃)₃ | C(CH₃)₃ | CD₃ | D | D | H₇ |
| 132 | C(CH₃)₃ | C(CH₃)₃ | CD₃ | H | D | H₇ |
| 133 | C(CH₃)₃ | C(CH₃)₃ | CD₃ | H | H | D₇ |
| 134 | C(CH₃)₃ | C(CH₃)₃ | CD₃ | H | H | H₇ |
| 135 | C(CH₃)₃ | C(CH₃)₃ | CD₃ | D | H | H₇ |
| 136 | C(CD₃)₃ | C(CH₃)₃ | CH₃ | D | D | D₇ |
| 137 | C(CD₃)₃ | C(CH₃)₃ | CH₃ | D | H | D₇ |
| 138 | C(CD₃)₃ | C(CH₃)₃ | CH₃ | H | D | D₇ |
| 139 | C(CD₃)₃ | C(CH₃)₃ | CH₃ | D | D | H₇ |
| 140 | C(CD₃)₃ | C(CH₃)₃ | CH₃ | H | D | H₇ |
| 141 | C(CD₃)₃ | C(CH₃)₃ | CH₃ | H | H | D₇ |
| 142 | C(CD₃)₃ | C(CH₃)₃ | CH₃ | D | H | H₇ |
| 143 | C(CD₃)₃ | C(CH₃)₃ | CH₃ | H | H | H₇ |
| 144 | C(CH₃)₃ | C(CD₃)₃ | CH₃ | D | D | D₇ |
| 145 | C(CH₃)₃ | C(CD₃)₃ | CH₃ | D | H | D₇ |
| 146 | C(CH₃)₃ | C(CD₃)₃ | CH₃ | H | D | D₇ |
| 147 | C(CH₃)₃ | C(CD₃)₃ | CH₃ | D | D | H₇ |
| 148 | C(CH₃)₃ | C(CD₃)₃ | CH₃ | H | D | H₇ |
| 149 | C(CH₃)₃ | C(CD₃)₃ | CH₃ | H | H | D₇ |
| 150 | C(CH₃)₃ | C(CD₃)₃ | CH₃ | D | H | H₇ |
| 151 | C(CH₃)₃ | C(CD₃)₃ | CH₃ | H | H | H₇ |
| 152 | C(CH₃)₃ | C(CH₃)₃ | CH₃ | D | D | D₇ |
| 153 | C(CH₃)₃ | C(CH₃)₃ | CH₃ | D | H | D₇ |
| 154 | C(CH₃)₃ | C(CH₃)₃ | CH₃ | H | D | D₇ |
| 155 | C(CH₃)₃ | C(CH₃)₃ | CH₃ | D | D | H₇ |
| 156 | C(CH₃)₃ | C(CH₃)₃ | CH₃ | H | D | H₇ |
| 157 | C(CH₃)₃ | C(CH₃)₃ | CH₃ | H | H | D₇ |
| 158 | C(CD₃)₃ | C(CD₃)₃ | CD₃ | H | D | D₇ |

or a pharmaceutically acceptable salt of any of the foregoing.

Alternative derivatives of boceprevir include Compounds 103 and 109 or pharmaceutically acceptable salts thereof.

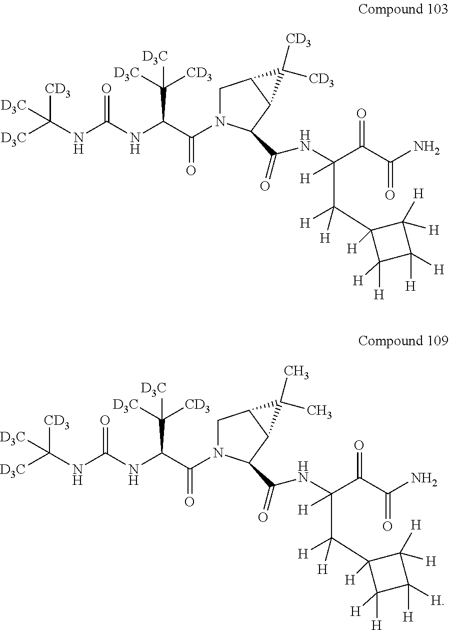

In yet another embodiment, the compound is selected from:

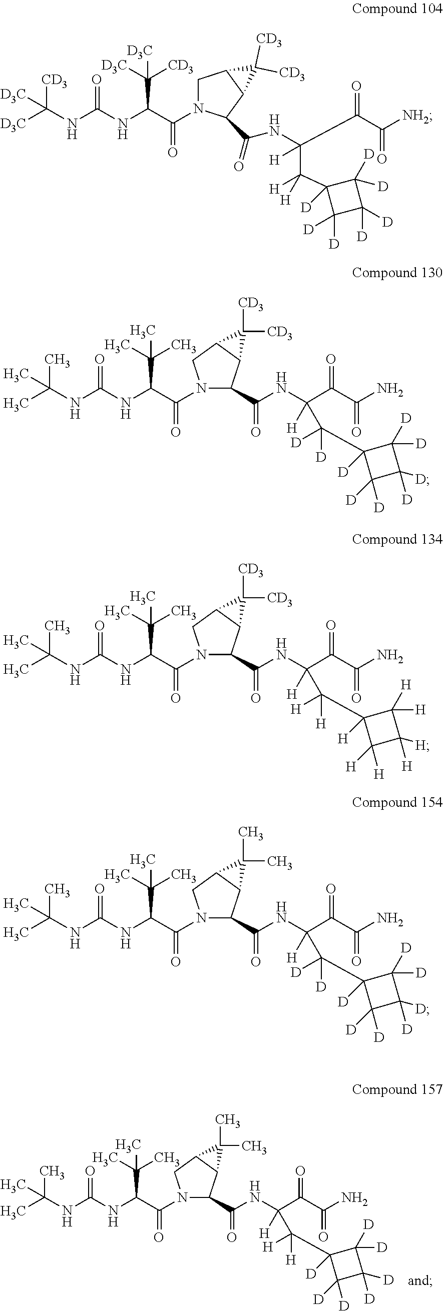

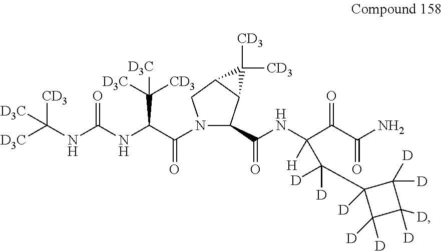

or a pharmaceutically acceptable salt of any of the foregoing.

In another set of embodiments, the configuration at chiral center 5 in any of the embodiments set forth above is from 50-100% “S.” In one aspect of this set of embodiments, the configuration at chiral center 5 is from about 60-80% “S.”

In yet another set of embodiments, the configuration at chiral center 5 in any of the embodiments set forth above is from 0-50% “S.” In one aspect of this set of embodiments, the configuration at chiral center 5 is from about 20-40% “S.”

In yet another set of embodiments, the configuration at each of chiral centers 1-4 is at least 92% of that depicted. In one aspect of this set of embodiments, the configuration at chiral center 5 is from 50-100% “S.” In a more specific aspect of this set of embodiments, the configuration at chiral center 5 is from about 60-80% “S.” In yet another aspect of this set of embodiments, the configuration at chiral center 5 is from 0-50% “S.” In a more specific aspect of this set of embodiments, the configuration at chiral center 5 is from about 20-40% “S.”

In yet another set of embodiments, the configuration at each of chiral centers 1-4 is at least 95% of that depicted. In one aspect of this set of embodiments, the configuration at chiral center 5 is from 50-100% “S.” In a more specific aspect of this set of embodiments, the configuration at chiral center 5 is from about 60-80% “S.” In yet another aspect of this set of embodiments, the configuration at chiral center 5 is from 0-50% “S.” In a more specific aspect of this set of embodiments, the configuration at chiral center 5 is from about 20-40% “S.”

In a further set of embodiments, any atom not designated as deuterium in any of the embodiments set forth above is present at its natural isotopic abundance.

The synthesis of compounds of Formula A can be readily achieved by synthetic chemists of ordinary skill. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in PCT publication WO 2004/113294, United States Patent publication US20070004635, or in Venkatraman, S et al., J Med Chem, 2006, 49: 6074. The compounds may be prepared generally as illustrated in the schemes shown below. The preparation of certain specific compounds is further described in the Examples below. It will be readily apparent to one skilled in the art that other specific compounds shown above may be prepared in an analogous manner by reference to the schemes and Examples.

### Exemplary Synthesis

A convenient method for synthesizing compounds of Formula A is depicted in Scheme 1 below.

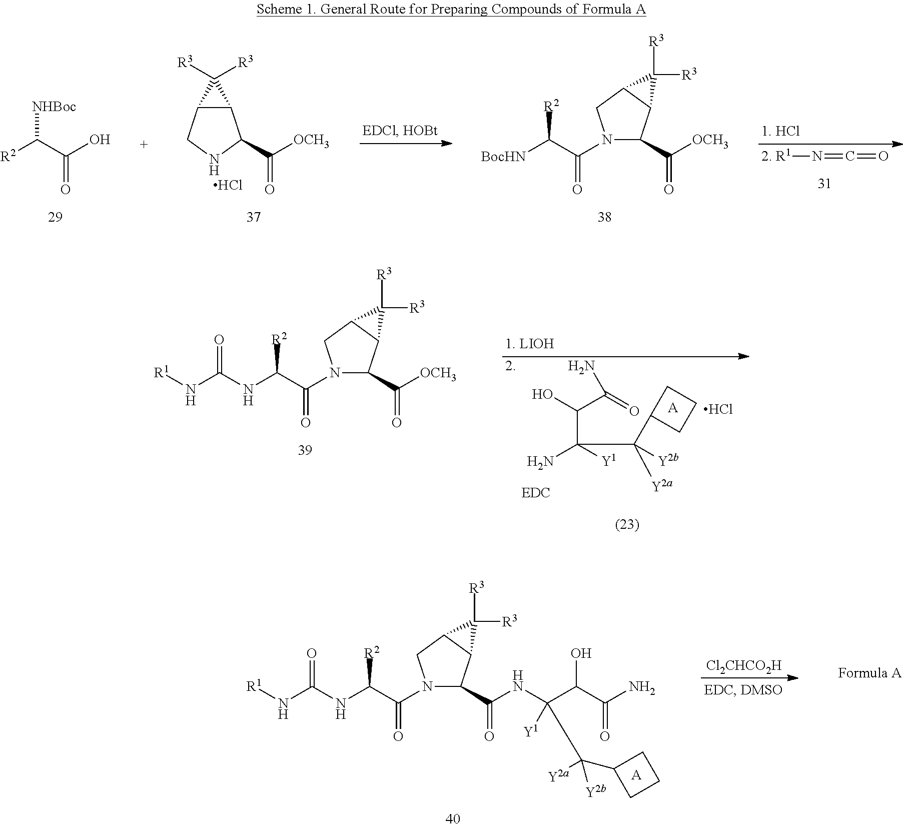

Scheme 1 above outlines a general route for preparing compounds of Formula A. Deuterated 3,4-dimethylcyclopropylproline 37 may be condensed with deuterated N-Boc-tert-leucine reagent 29 using the procedures described by Venkatraman, S et al., J Med Chem, 2006, 49: 6074-6086 to afford the dipeptide 38. Acidic removal of the Boc group with HCl and coupling of the corresponding amine with deuterated tert-butylisocyanate 31 (prepared from the correspondingly deuterated amine R¹—NH₂ 30 (such as t-butylamine-d₉, 99 atom % D from CDN Isotopes) by treatment with hydrochloric acid and then triphosgene) affords the dipeptide 39. Following LiOH hydrolysis of 39, its corresponding carboxylic acid may be coupled to the alpha-hydroxy amide 23 to produce hydroxyamide 40, which then may be oxidized to afford a compound of Formula A.

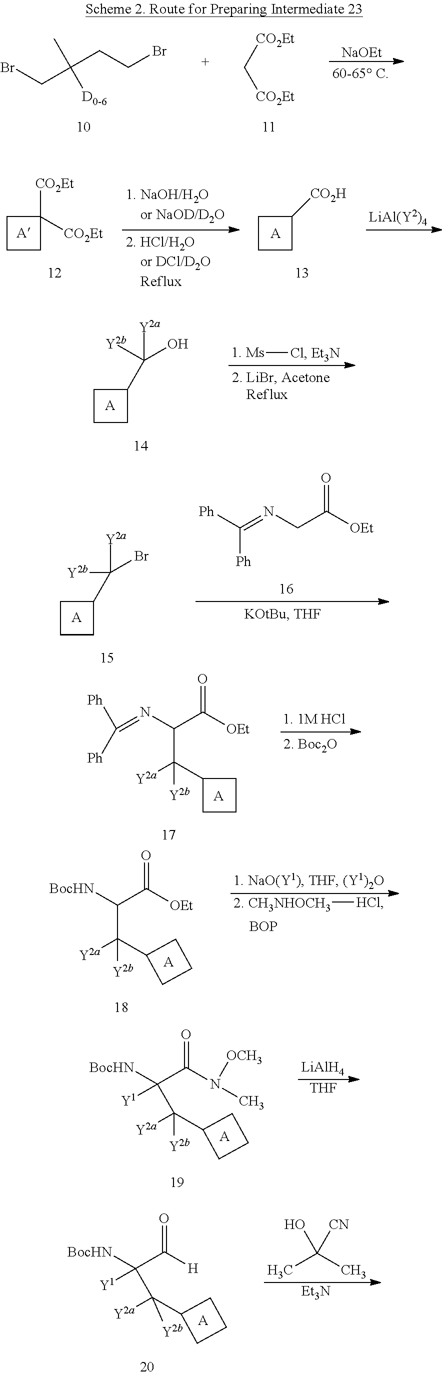

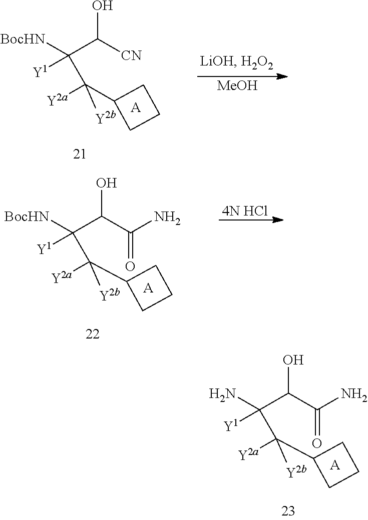

Scheme 2 outlines a route for preparing appropriately deuterated cyclobutylmethyl alpha-hydroxyamide 23, for use in Scheme 1. Treatment of an appropriately deuterated dibromopropane 10 (such as commercially available dibromopropane-d₆, 99 atom % D from CDN Isotopes) with diethylmalonate 11 in the presence of sodium ethoxide using the procedure described by Heisig, G B et al., Org Synth, 1955, 3: 213-215 affords the corresponding appropriately deuterated ethyl-1,1-cyclobutanedicarboxylate 12, where A′ is a cyclobutyl group substituted with 0-6 deuterium. Saponification of the ethyl ester moieties in 12 with sodium hydroxide or sodium deuteroxide (99.5 atom % D from Aldrich) followed by decarboxylation with hydrochloric acid or deuterium chloride (99.9 atom % D from Cambridge Isotopes) using the procedures in the aforementioned reference affords deuterated cyclobutanecarboxylic acid 13, where A is a cyclobutyl group substituted with 0-7 deuterium. Reduction of 13 with lithium aluminum deuteride 96 atom % D from Aldrich) or lithium aluminum hydride (LiAl(Y²)₄) using the procedure described by Ingold, K U et al., J Chem Soc, Perkin Trans II, 1981, 970-974 affords the appropriately deuterated cyclobutylmethanol 14. Subsequent activation of the alcohol moiety in 14 as the corresponding mesylate and displacement with lithium bromide using the procedure from Ingold affords the corresponding cyclobutylmethyl bromide 15.

The cyclobutylmethyl bromide 15 then may be combined with the potassium enolate of ethyl N-(diphenylmethylene)glycinate 16 (generated in situ by treatment of 16 with potassium tert-butoxide) using the protocol described by Venkatraman, S et al., J Med Chem, 2006, 49: 6074-6086 to afford the corresponding glycine derivative 17. Hydrolysis of the diphenylimine moiety in 17 followed by Boc protection of the amine produces the Boc-protected amino ester 18. The Y¹ group may be incorporated by treatment of 18 with either sodium hydroxide or sodium deuteroxide (NaOY¹), followed by conversion of the resulting acid to the corresponding Weinreb amide 19 upon treatment with N,O-dimethylhydroxylamine and benzotriazole-1-yl-oxy-tris-(dimethylamino)-phosphonium hexafluorophosphate (BOP). Conversion of amide 19 to the corresponding aldehyde 20 may be achieved by treatment with LiAlH₄ in THF. Aldehyde 20 may be treated with acetone cyanohydrin in triethylamine to produce cyanohydrin 21. Hydrolysis of cyanohydrin 21 to hydroxyamide 22 may be accomplished by treatment with LiOH and hydrogen peroxide. This may be followed by acid catalyzed cleavage of the Boc group in 22 to form a deuterated alpha hydroxy amide 23.

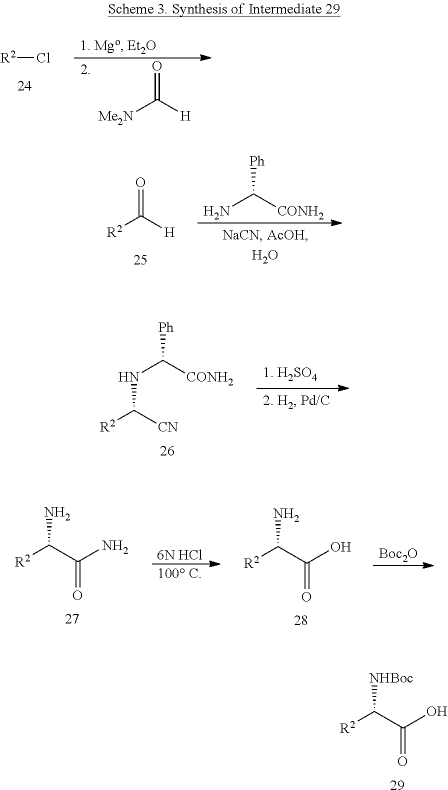

Scheme 3 above depicts one synthetic pathway to the deuterated N-Boc-tert-leucine intermediate 29, useful in Scheme 1. A deuterated Grignard reagent may be generated in situ by reaction of a deuterated 2-chloro-2-methylpropane 24 (such as commercially available 2-chloro-2-methylpropane-d₉, 98 atom % D from Cambridge Isotopes) with magnesium metal. The Grignard reagent may be treated with N,N-dimethylformamide according to the procedures described by Nazarski, R B et al., Bull Soc Chim Belg, 1992, 101: 817-819 to afford the corresponding pivaldehyde 25. Treatment of the pivaldehyde 25 with (R)-phenylglycine amide, followed by reaction of the corresponding chiral imine with sodium cyanide according to the procedure described by Boesten, W H et al., Org Lett 2001, 3: 1121-1124, affords the alpha-amino nitrile 26. Hydrolysis of nitrile 26 to the diamide followed by hydrogenolysis of the phenylacetamide group gives the alpha-amino amide 27. Finally, hydrolysis of the tert-leucine amide with 6N HCl gives the corresponding acid 28, which then may be Boc-protected to form the desired N-Boc-tert-leucine reagent 29.

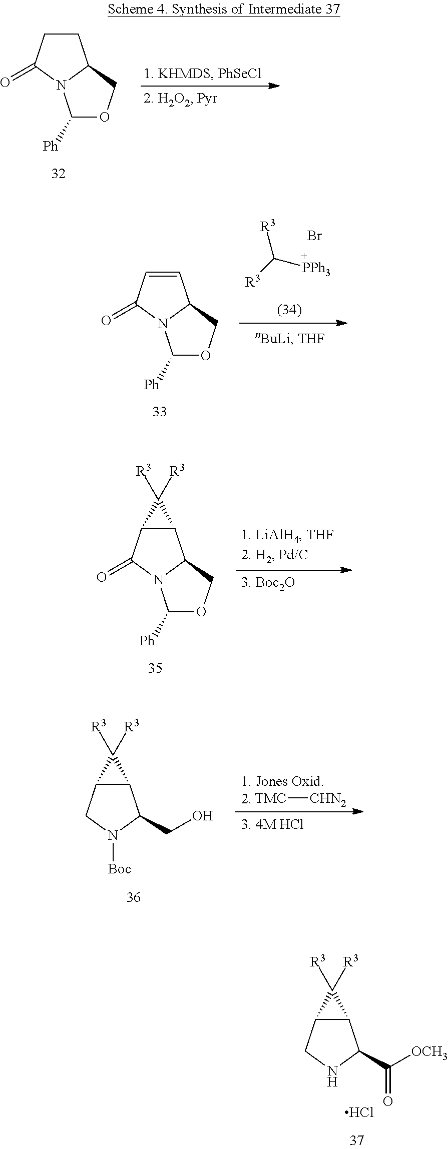

Scheme 4 depicts a synthetic route to deuterated 3,4-dimethylcyclopropylproline 37, useful in Scheme 1. Treatment of the potassium enolate of (3R,7aS)-tetrahydro-3-phenyl-3H,5H-pyrrolo1,2-coxaole-5-one (generated in situ by reaction of commercially available (3R,7aS)-tetrahydro-3-phenyl-3H,5H-pyrrolo-1,2-coxaole-5-one 32 with potassium hexamethyldisilazane) with phenyl selenium chloride followed by oxidation and elimination of the selenoxide according to the procedures described by Madalengoitia, J S et al., J Org Chem, 1999, 64: 547-555 affords the alpha, beta-unsaturated lactam 33. Treatment of the lactam 33 with a deuterated isopropylphosphonium ylide 34 (prepared in situ by from a deuterated isopropyl bromide (such as commercially available d₇-isopropyl bromide, 98 atom % D from Aldrich), see Braverman, S et al., J Am Chem Soc, 1990, 112: 5830-5837) using the procedure described by Ahmad, S et al., J Med Chem, 2001, 44: 3302-3310 affords the corresponding dimethylcyclopropyl lactam 35. Lactam 35 may be converted to the requisite cyclopropylproline methyl ester 37 following the method described by Venkatraman, S et al., J Med Chem, 2006, 49: 6074-6086.

A number of novel intermediates are described herein that can be used to prepare compounds of Formula A. Such compounds that are useful intermediates include those represented by Formula H:

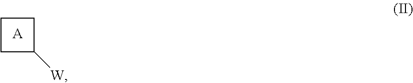

wherein:

Ring A is a cyclobutyl group substituted by 1 to 7 deuterium atoms;

W is CO₂R⁰ or C(Y³)₂Z;

R⁰ is hydrogen, deuterium or a C₁₋₆ alkyl group;

each Y³ is independently selected from hydrogen and deuterium;

Z is selected from OH, Br,

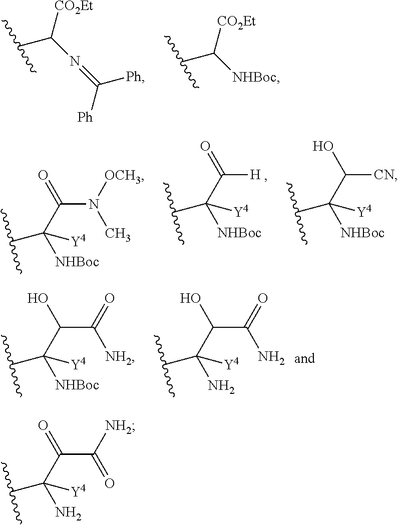

and

Y⁴ is hydrogen or deuterium.

In compounds of Formula II, Ring A preferably has 6 or 7 deuterium atoms and is fully deuterated at positions 2, 3 and 4 of the cyclobutyl ring (where the carbon bearing the W group is position 1). Examples of R⁰ include hydrogen, deuterium, methyl and ethyl. Each Y³ is preferably the same.

The invention also provides other compounds that are useful in preparing compounds of Formula A, including the following:

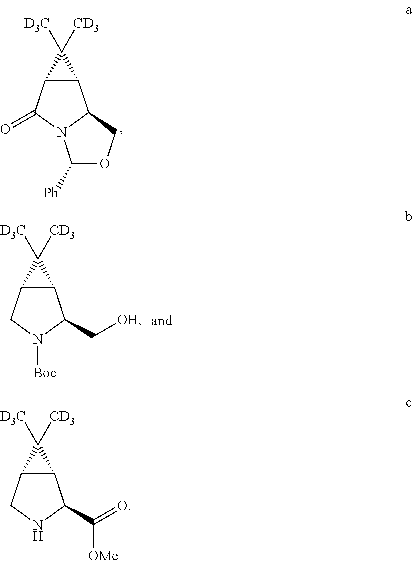

Compounds of Formula II above may be prepared as outlined in Scheme 2 using 1,3-dibromopropane-d₆ as the starting material 10 and appropriately deuterated reagents for the decarboxylation and reduction steps shown. Compounds of Formula II where W is C(Y³)₂Z and Z is

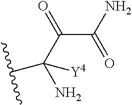

may be prepared by subsequent Swern oxidation of the α-hydroxyamide. Compounds “a-c” above may be prepared according to Scheme 4 using isopropyl bromide-d₇ to generate the phosphonium ylide “a”.

Under certain synthetic conditions, Compounds 130, 134, 154, 157 and 158 may be prepared with an isotopic abundance at each position indicated as “D” of at least about 75%. Under other synthetic conditions Compounds 130, 134, 154, 157 and 158 may be prepared with an isotopic abundance at the 1 position of the butanyl portion (methylene alpha to the cyclobutyl ring) indicated as “D” of greater than about 90% and an isotopic abundance at all other positions indicated as “D” of greater than about 95%.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula A and their synthetic precursors, including those within routes not explicitly shown in schemes herein, are within the means of chemists of ordinary skill in the art. Methods for optimizing reaction conditions and, if necessary, minimizing competing by-products, are known in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al, Protective Groups in Organic Synthesis, 3^{rd} Ed., John Wiley and Sons (1999); Fieser L et al, Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula A (e.g., including any of the formulae herein), or a pharmaceutically acceptable salt of said compound; and a pharmaceutically acceptable carrier acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as boceprevir. Such agents include those indicated as being useful in combination with boceprevir, including but not limited to, those described in WO 2006130666, WO 2006130628, WO 2007092616, and WO 2007092645.

Preferably, the second therapeutic agent is an agent useful in the treatment or prevention of a disease or condition selected from disorders associated with hepatitis C virus (HCV).

In one embodiment, the second therapeutic agent is selected from PEG-interferon alpha-2a, PEG-interferon alpha-2b, ribavirin, telapravir, nitazoxanide and combinations of two or more of the foregoing.

In a more specific embodiment, the second therapeutic agent is a combination of PEG-interferon alpha-2a and ribavirin.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al, (1966) Cancer Chemother Rep 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from about 1 mg to about 8000 mg per treatment. In a more specific embodiment the range is from about 10 to 4000 mg, or from 20 to 1600 mg, or most specifically, from about 100 to 800 mg per treatment. Treatment typically is administered from one to three times daily.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the dosages of boceprevir being utilized in clinical trials.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of blocking the activity of HCV NS3/NS4A protease in an infected cell, comprising contacting such a cell with one or more compounds of Formula A herein.

For each of the methods of treatment disclosed herein, the subject being treated may be, for example, a patient in need of the treatment.

According to another embodiment, the invention provides a method of treating a disease that is beneficially treated by boceprevir in a subject comprising the step of administering to said subject an effective amount of a compound or a composition of this invention. Such diseases are well known in the art and are disclosed in, but not limited to the following patents and published applications: WO 2002008244, and WO 2003062265. Such diseases include, but are not limited to, disorders associated with hepatitis C virus (HCV).

In one particular embodiment, the method of this invention is used to treat a hepatitis C viral (HCV) infection in a subject.

Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to said subject one or more second therapeutic agents. The choice of second therapeutic agent may be made from any second therapeutic agent known to be useful for co-administration with boceprevir. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula A and a second therapeutic agent for treatment of the following conditions (with the particular second therapeutic agent indicated in parentheses following the indication: hepatitis C (PEG-interferon, and ribavirin). (See clinical trials for SCH-503034 at http://clinicaltrials.gov/).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula A alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of Formula A for use in the treatment or prevention in a subject of a disease, disorder or symptom thereof delineated herein.

### Diagnostic Methods and Kits

The present invention also provides kits for use to treat hepatitis C viral infection. These kits comprise (a) a pharmaceutical composition comprising a compound of Formula A or a salt thereof, wherein said pharmaceutical composition is in a container; and (b) instructions describing a method of using the pharmaceutical composition to treat hepatitis C viral infection.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

### EXAMPLES

### Example 1

### Synthesis of 3-Amino-4-(cyclobutyl-d₇)-4,4-d₂-2-oxobutanamide hydrochloride (82)

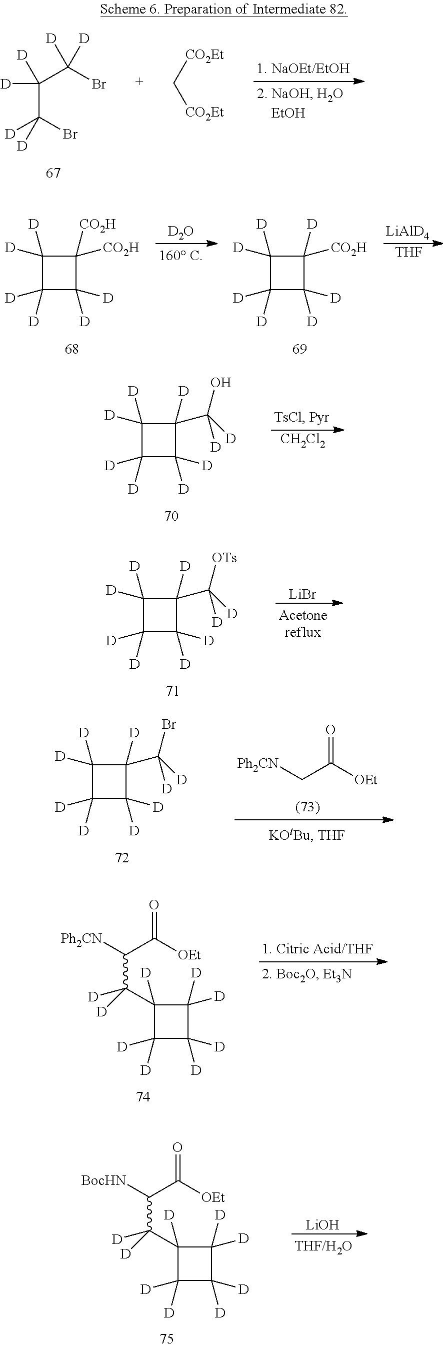

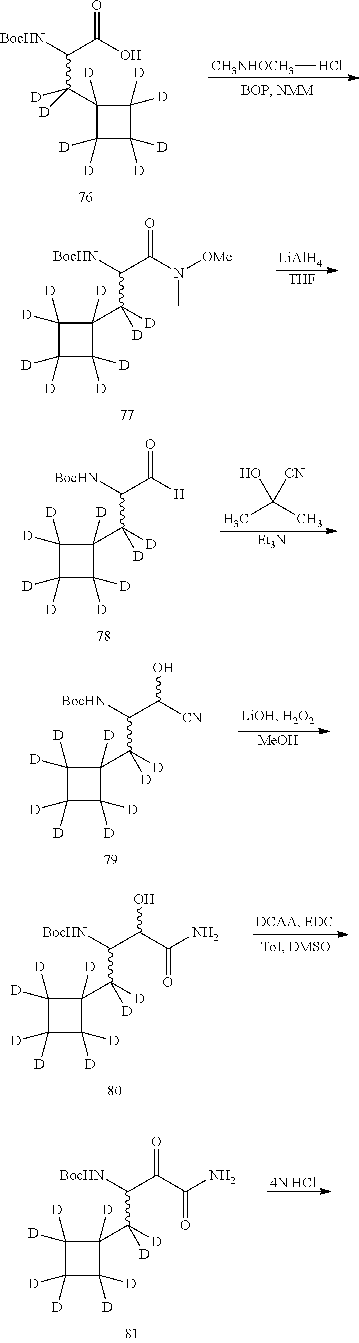

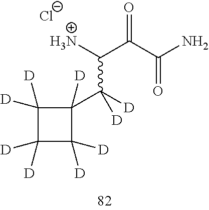

### Step 1. 2,2,3,3,4,4-d₆-Cyclobutane-1,1-dicarboxylic acid (68)

To a stirred solution of 67 (3.54 mL, 33.7 mmol, 99 atom % D, CDN Isotopes) and diethylmalonate (4.87 mL, 32.1 mmol) in ethanol (35 mL) at 60-65° C., was added dropwise a 21 wt % solution of sodium ethoxide in ethanol (24.1 mL, 64.2 mmol). Upon completion of the addition the reaction was cooled to approximately 50° C. and was subsequently stirred at 100° C. until an aliquot added to water was neutral to pH paper. The reaction was then cooled to room temperature, diluted with water and concentrated in vacuo to remove ethanol. The resulting aqueous solution was extracted with EtOAc (3×100 mL), the combined organics dried (Na₂SO₄), filtered and concentrated in vacuo. The resulting residue was purified by column chromatography (SiO₂, 0-20% EtOAc/heptane) to afford the diethyl ester derivative of 68 (4.00 g, 60% yield). ¹H NMR (CDCl₃, 400 MHz) δ 4.20 (q, 2H, J=7.1 Hz), 1.25 (t, 3H, J=7.1 Hz). To a solution of this diester (4.00 g, 19.4 mmol) in ethanol (10 mL) was added 5M aqueous sodium hydroxide (9.00 mL, 45.0 mmol). The mixture was heated to reflux and stirred for 2 hours. Upon cooling to room temperature the reaction mixture was concentrated in vacuo and diluted with 6N HCl (100 mL). The resulting aqueous solution was extracted with diethyl ether (3×100 mL) and the combined organic layers were dried (Na₂SO₄), filtered and concentrated in vacuo. The resulting residue was recrystallized from EtOAc/heptane to afford 68 as a white crystalline solid (2.21 g, 76%). This material was carried forward into the next step.

### Step 2. (Cyclobutane-d₇)carboxylic acid (69)

A solution of dicarboxylic acid 68 (2.21 g, 14.7 mmol) in D₂O (30 mL, 99.9 atom % D, Cambridge Isotope Laboratories) in a sealed pressure tube was heated to 160° C. for 15 hours. After cooling to room temperature the reaction mixture was diluted with excess 1M HCl and extracted with EtOAc (3×100 mL). The combined organic layers were dried (Na₂SO₄), filtered and concentrated in vacuo to afford 69 (1.57 g, 100%) as a clear oil which formed colorless crystals upon cooling in a −20° C. freezer. This material was carried forward into the next step.

### Step 3. (Cyclobutyl-d₇)-1,1-d₂-methanol (70)

To a solution of 69 (1.57 g, 14.7 mmol) in THF (65 mL) at 0° C. was added a 1M solution of LiAlD₄ in THF (20 mL, 20 mmol, 96 atom % D, Aldrich). The reaction was stirred at room temperature for 4 hours, cooled to 0° C. and quenched by dropwise addition of 10% KHSO₄ until a solid white cake formed and further addition of KHSO₄ caused no visible reaction. The cake was broken up, filtered through Celite and washed with diethyl ether repeatedly. The resulting filtrate was concentrated in vacuo, dissolved in dichloromethane (100 mL), dried (Na₂SO₄), filtered and concentrated in vacuo to afford 70 as a clear oil (1.27 g, 91%). This material was carried forward into the next step.

### Step 4. (Cyclobutylmethyl-d₉) 4-methylbenzenesulfonate (71)

To a solution of 70 (1.27 g, 13.4 mmol) in dichloromethane (25 mL) at 0° C. was added pyridine (3.25 mL, 40.2 mmol) followed by p-toluenesulfonyl chloride (2.56 g, 13.4 mmol). The reaction was stirred at room temperature for 15 hours then was diluted with diethyl ether (200 mL). The resulting solution was washed with water (100 mL), 1M HCl (3×50 mL), water (100 mL), and brine (100 mL). The organic layer was dried (MgSO₄), filtered and concentrated in vacuo to afford 71 as a clear oil (2.28 g, 68%). ¹H NMR (CDCl₃, 400 MHz): δ 7.78 (d, 2H, J=8.3 Hz), 7.34 (d, 2H, J=8.3 Hz), 2.45 (s, 3H). MS (M+Na): 272.1.

### Step 5. (Bromomethyl)cyclobutane-d₉ (72)

A solution of tosylate 71 (2.28 g, 9.14 mmol) and lithium bromide (1.27 g, 14.6 mmol) in acetone (30 mL) was heated to reflux and stirred for 3 hours. The reaction was then cooled, diluted with diethyl ether, and filtered through Celite to remove lithium tosylate. The acetone and ether were removed via careful distillation (note: bromomethylcyclobutane bp=123° C.). The resulting residue was dissolved in ether (50 mL) and the solution was washed with water (2×50 mL), dried (MgSO₄), and filtered. Diethylether was then removed via distillation to afford 72 as a clear oil containing diethyl ether (1.55 g total, ˜1.16 g of 7, ˜80%). This material was carried forward into the next step.

### Step 6. Ethyl 3-(Cyclobutyl-d₇)-2-(diphenylmethyleneamino)-3,3-d₂-propanoate (74)

To a solution of ethyl 2-(diphenylmethyleneamino)acetate, 73 (1.51 g, 5.65 mmol) in THF (13 mL) at −78° C. was added a 1M solution of potassium tert-butoxide in THF (6.78 mL, 6.78 mmol). The resulting solution was stirred at 0° C. for 1 hour at which time a solution of bromide 72 (1.16 g, 7.34 mmol) in THF (2 mL) was added. The reaction mixture was then stirred at room temperature for 15 hours. THF was removed in vacuo, the resulting residue was dissolved in water (100 mL) and this solution was extracted with dichloromethane (3×50 mL). The aqueous layer was acidified with 1M HCl and re-extracted with dichloromethane (3×50 mL). All organic layers were combined, dried (Na₂SO₄), filtered and concentrated in vacuo to afford a red oil which was purified by column chromatography (SiO₂, 0-20% EtOAc/heptane) to afford ethyl ester 74 (704 mg, 36% yield). ¹H NMR (CDCl₃, 400 MHz): δ 7.66-7.61 (m, 2H), 7.49-7.29 (m, 6H), 7.20-7.14 (m, 2H), 4.23-4.09 (m, 2H), 3.97 (s, 1H), 1.26 (t, 3H, J=7.1 Hz). MS (M+H): 345.2.

### Step 7. Ethyl 2-(Tert-butoxycarbonylamino)-3-(cyclobutyl-d₇)-3,3-d₂-propanoate (75)

A 15% citric acid solution (10 mL) was added to a stirred solution of 74 (704 mg, 2.04 mmol) in THF (10 mL) at room temperature. The reaction mixture was stirred for 3 hours then THF was removed in vacuo. The resulting aqueous solution was quenched with saturated NaHCO₃ and extracted with EtOAc (3×50 mL). The combined organic layers were dried (Na₂SO₄), filtered and concentrated in vacuo to afford a 1:1 mixture of cyclobutylalanine ethyl ester-d9 and benzophenone. This material was dissolved in dichloromethane (8 mL) and triethylamine (427 μL, 3.06 mmol) was added followed by di-tert-butyl dicarbonate (535 mg, 2.45 mmol). The reaction mixture was stirred for 15 hours then was diluted with 1M HCl and extracted with dichloromethane (3×50 mL). The combined organic layers were washed with saturated NaHCO₃ and brine, dried (MgSO₄), filtered and concentrated in vacuo. The resulting residue was purified by column chromatography (SiO₂, 0-20% EtOAc/heptane) to afford 75 (436 mg, 76% yield, 2 steps). ¹H NMR (CDCl₃, 400 MHz): δ 4.94 (br s, 1H), 4.20-4.13 (m, 3H), 1.43 (s, 9H), 1.28 (t, 3H, J=7.1 Hz). MS (M+Na): 303.2; (M-Boc+H): 181.2.

### Step 8. 2-(Tert-butoxycarbonylamino)-3-(cyclobutyl-d₇)-3,3-d₂-propanoic acid (76)

Lithium hydroxide (56.0 mg, 2.33 mmol) was added to a solution of 75 (436 mg, 1.55 mmol) in 1:1 THF/H₂O (14 mL). The reaction mixture was stirred for 15 hours at which time THF was removed in vacuo. The resulting aqueous solution was diluted with 1M HCl and extracted with EtOAc (3×50 mL). The combined organic layers were washed with brine, dried (Na₂SO₄), filtered and concentrated in vacuo to afford 76 (381 mg, 97%). MS (M+Na): 275.3; (M-Boc+H): 153.2.

### Step 9. Tert-butyl 3-(Cyclobutyl-d₇)-1-(methoxy(methyl)amino)-3,3-d₂-1-oxopropan-2-ylcarbamate (77)

To a solution of 76 (381 mg, 1.51 mmol) in dichloromethane (7 mL) at 0° C. was added N,O-dimethylhydroxylamine hydrochloride (221 mg, 2.26 mmol) followed by BOP reagent (1.00 g, 2.26 mmol) and N-methyl morpholine (664 μL, 6.04 mmol). The reaction mixture was stirred at room temperature for 15 hours then was diluted with dichloromethane (100 mL), washed with 1M HCl (3×50 mL), dried (Na₂SO₄), filtered and concentrated in vacuo. The resulting residue was purified by column chromatography (SiO₂, 10-30% EtOAc/heptane) to afford the Weinreb amide 77 (336 mg, 75%) as a white solid. ¹H NMR (CDCl₃, 400 MHz): δ 5.10 (br d, 1H, J=9.1 Hz), 4.55 (br d, 1H, J=8.8 Hz), 3.75 (s, 3H), 3.17 (s, 3H), 1.41 (s, 9H). MS (M+Na): 318.3; (M-Boc+H): 196.2.

### Step 10. Tert-butyl 1-(Cyclobutyl-d₇)-1,1-d₂-3-oxopropan-2-ylcarbamate (78)

A 2M solution of LiAlH₄ in THF (1.02 mL, 2.05 mmol) was added dropwise to a solution of 77 (336 mg, 1.14 mmol) in THF (5 mL) at 0° C. The reaction mixture was stirred at room temperature for 1 hour, was cooled to 0° C. and the reaction was quenched by dropwise addition of 10% KHSO₄ until a solid white cake formed and further addition of KHSO₄ was caused no visible reaction. The cake was broken up, filtered through Celite and washed with EtOAc repeatedly. The resulting filtrate was concentrated in vacuo, diluted with water (50 mL) and extracted with EtOAc (3×50 mL). The combined organic layers were dried (Na₂SO₄), filtered and concentrated in vacuo to afford 78 as a colorless solid (221 mg, 82%). ¹H NMR (CDCl₃, 400 MHz): δ 9.55 (s, 1H), 5.00 (br s, 1H), 4.15 (br d, 1H, J=7.1 Hz), 1.44 (s, 9H). MS (M+Na): 259.3; (M-Boc+H): 137.3.

### Step 11. Tert-butyl 1-Cyano-3-(cyclobutyl-d₇)-3,3-d₂-1-hydroxypropan-2-ylcarbamate (79)

To a solution of aldehyde 78 (221 mg, 0.935 mmol) and triethylamine (158 μL, 1.13 mmol) in dichloromethane (1 mL) was added acetone cyanohydrin (177 μL, 1.94 mmol). The reaction mixture was stirred at room temperature for 15 hours then was concentrated in vacuo. The resulting residue was diluted with 1M HCl (10 mL) and extracted with dichloromethane (3×30 mL). The organic layers were combined, washed with water then brine, dried (MgSO₄), filtered and concentrated in vacuo. The resulting residue was purified by column chromatography (SiO₂, 0-20% EtOAc/heptane) to afford 79 (215 mg, 87%) as a clear oil and as a mixture of diastereomers. MS (M+Na): 286.2; (M-Boc+H): 164.2.

### Step 12. Tert-butyl 4-Amino-1-(cyclobutyl-d₇)-3-hydroxy-1,1-d₂-4-oxobutan-2-ylcarbamate (80)

To a solution of cyanohydrin 79 (215 mg, 0.816 mmol) in methanol (3 mL) at 0° C. was added 30% H₂O₂ (509 μL, 4.49 mmol) followed by lithium hydroxide (24.0 mg, 0.980 mmol). The reaction mixture was stirred at 0° C. for 3 hours then was quenched at 0° C. via careful addition of excess saturated NaHSO₃. The resulting solution was diluted with water (20 mL) and extracted with dichloromethane (3×30 mL). The combined organic layers were dried (Na₂SO₄), filtered and concentrated in vacuo to afford 80 (141 mg, 61%) as a white solid. MS (M+Na): 304.3; (M-Boc+H): 182.3.

### Step 13. Tert-butyl 4-Amino-1-(cyclobutyl-d₇)-1,1-d₂-3,4-dioxobutan-2-ylcarbamate (81)

To a solution of amide 80 (141 mg, 0.501 mmol) in 1:1 toluene/DMSO (10 mL) at 0° C. was added 1-ethyl-3-(3-dimethylaminopropyl) carbodiimide hydrochloride (EDC) (960 mg, 5.01 mmol) followed by dichloroacetic acid (DCAA) (207 μL, 2.50 mmol). The reaction mixture was stirred at room temperature for 4 hours then was diluted with 1M HCl (50 mL) and extracted with dichloromethane (3×50 mL). The combined organic layers were washed with 1M HCl (50 mL), saturated NaHCO₃ (50 mL) and brine (50 mL), dried (Na₂SO₄), filtered and concentrated in vacuo. The resulting residue was purified by column chromatography (SiO₂, 0-40% EtOAc/heptane) to afford 81 (117 mg, 84%) as an off-white solid. MS (M+Na): 302.2; (M-Boc+H): 180.2.

### Step 14. 3-Amino-4-(cyclobutyl-d₇)-4,4-d₂-2-oxobutanamide hydrochloride (82)

Ketoamide 81 (117 mg, 0.419 mmol) was stirred in 4M HCl in dioxane (7 mL) for 3 hours. The reaction mixture was then concentrated in vacuo to afford 82 (90 mg, 99%. This material was carried forward without purification.

### Example 2

### Synthesis of (1R,2S,5S)—N-(4-Amino-1-(cyclobutyl-d₇)-1,1-d₂-3,4-dioxobutan-2-yl)-3-((S)-2-(3-tert-butylureido)-3,3-dimethylbutanoyl)-6,6-dimethyl-3-azabicyclo[3.1.0]hexane-2-carboxamide (Compound 154).

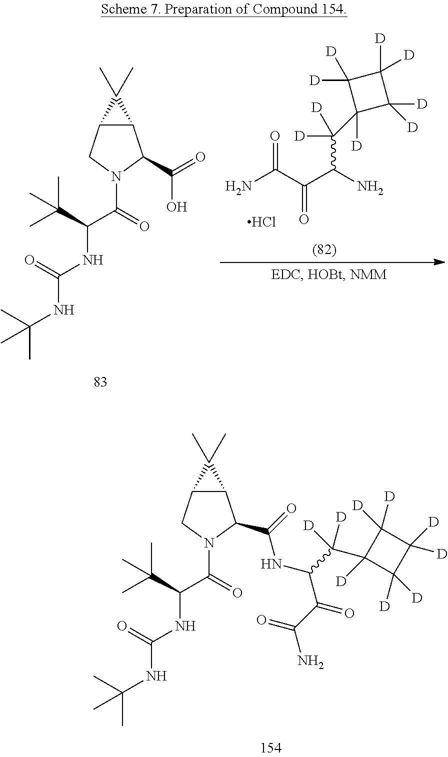

### (1R,2S,5S)—N-(4-Amino-1-(cyclobutyl-d₇)-1,1-d₂-3,4-dioxobutan-2-yl)-3-((S)-2-(3-tert-butylureido)-3,3-dimethylbutanoyl)-6,6-dimethyl-3-azabicyclo[3.1.0]hexane-2-carboxamide (Compound 154)

To a solution of carboxylic acid 83 (59.0 mg, 0.160 mmol, see J. Med. Chem., 2006, 49: 6074-6086 for preparation) and amine hydrochloride 82 (45.0 mg, 0.209 mmol, see Example 1) in acetonitrile (1.5 mL) at 0° C. was added EDC (46.0 mg, 0.240 mmol), HOBt (6.00 mg, 0.0480 mmol) and N-methyl morpholine (19.0 μL, 0.176 mmol). The reaction mixture was stirred at room temperature for 15 hours, concentrated in vacuo, diluted with 1M HCl, and extracted with EtOAc (3×20 mL). The combined organic layers were washed with 1M HCl, saturated NaHCO₃ and brine, dried (MgSO₄), filtered and concentrated in vacuo. The resulting residue was purified by column chromatography (SiO₂, 10-40% acetone/heptane) to afford Compound 154 with a dr=1.7:1 (S:R at chiral carbon 5) (21 mg, 25%) as a white solid. ¹H NMR (CDOD₃, 400 MHz): δ 7.55 (br s, 0.3H), 6.07 (br s, 0.6H), 5.95-5.76 (m, 1.3H), 4.38 (s, 0.6H), 4.35-4.20 (m, 2H), 4.13-3.86 (m, 3H), 3.25-3.20 (m, 1H), 1.62-1.27 (m, 2H), 1.29-1.22 (m, 9H), 1.08-1.03 (m, 3H), 1.03-0.96 (m, 9H), 0.95-0.87 (m, 3H). MS (M+H): 529.5.

### Example 3

### Synthesis of (1R,2S,5S)—N-(4-Amino-1-(cyclobutyl-d₇)-1,1-d₂-3,4-dioxobutan-2-yl)-3-((S)-2-(3-(tert-butyl-d₉)ureido)-3,3-di(methyl-d₃)-4,4,4-d₃-butanoyl)-6,6-di(methyl-d₃)-3-azabicyclo[3.1.0]hexane-2-carboxamide (Compound 158).

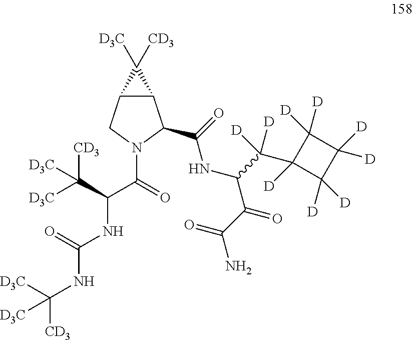

### Part a. Synthesis of (S)-2-(Tert-butoxycarbonylamino)-3,3-di(methyl-d₃)-4,4,4-d₃-butanoic acid (56)

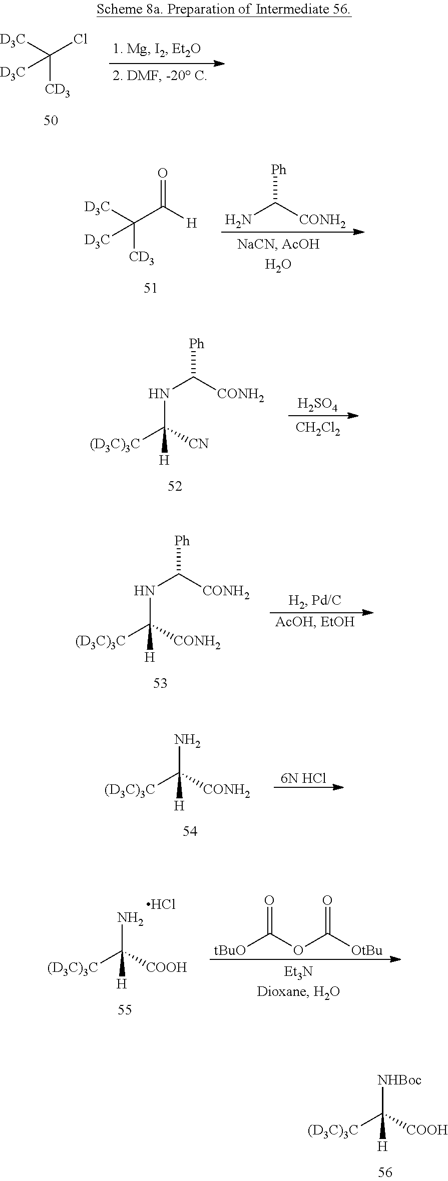

### Step 1. Pivalaldehyde-d₉ (51)

In a 3-L 4-necked round bottom flask fitted with mechanical stirrer, reflux condenser, dropping funnel and thermometer were placed a few small crystals of iodine and then magnesium turnings (24.7 g, 1.03 mol). The bottom of the flask was heated with a heat gun until the iodine commenced to vaporize. The flask was allowed to cool while a solution of t-butyl chloride-d₉ 50 (100.0 g, 1.03 mol, Cambridge Isotopes, 98% isotopic purity) in anhydrous ether was placed in the dropping funnel. A portion of the solution of 50 in ether (3-5 mL) was added directly to the dry magnesium. More anhydrous ether (1 L) and a few small crystals of iodine were added, and the resulting mixture was heated for 0.5 hours to initiate the reaction. The remainder of the solution of 50 in ether was added with stirring at a rate not faster than one drop per second. The mixture was allowed to reflux during the halide-ether addition and no external cooling was applied. The resulting mixture was heated at reflux for several hours until almost all magnesium had disappeared. The mixture was cooled to −20° C., and a solution of anhydrous DMF (73.0 g, 1.0 mol) in ether (100 mL) was added over a 35 minute period at such a rate that the temperature of the reaction did not exceed −15° C. A second solution of anhydrous DMF (73.0 g, 1.0 mol) was then added quickly at −8° C. After an additional 5 min, hydroquinone (0.5 g) was added, stirring was stopped, the cooling bath was removed, and the mixture was left standing overnight at room temperature under nitrogen. The mixture was cooled to 5° C. and aqueous 4M HCl (600 mL) was added in portions to quench the reaction. The resulting mixture was diluted with water (400 mL), and the layers were separated. The aqueous layer was extracted with ether (3×200 mL), and the combined organic layers were dried (Na₂SO₄) and filtered. The filtrate was subjected to fractional distillation under an atmosphere of nitrogen to remove most of the ether. The residue was transferred to a small flask and fractional distillation was continued to afford 39.5 g (40% yield) of the desired compound 51 as a colorless oil at 65-75° C. Compound 51 was stored under nitrogen in the freezer.

### Step 2. (R)-2-((S)-1-Cyano-2,2-di(methyl-d₃)-3,3,3-d₃-propylamino)-2-phenylacetamide (52)

To a stirred suspension of (R)-phenylglycine amide (60.7 g, 400 mmol) in water (400 mL) was added compound 51 (39.5 g, 415 mmol) at room temperature. This was followed by simultaneous addition of 30% aqueous NaCN solution (68.8 g, 420 mmol) and glacial acetic acid (25.4 g, 423 mmol) over 30 minutes, during which time the temperature of the reaction increased to 34° C. The mixture was stirred for 2 hours at 30° C., followed by stirring at 70° C. for 20 hours. After cooling to 30° C., the product was isolated by filtration. The solid was washed with water (500 mL) and dried under vacuum at 50° C. to afford the desired compound 52 (90.0 g, 88% yield) as a tan solid with [α]_{D}=−298° (c=1.0, CHCl₃).

### Step 3. (S)-2-((R)-2-Amino-2-oxo-1-phenylethylamino)-3,3-dimethyl-d₃)-4,4,4-d₃-butanamide (53)

A solution of compound 52 (64.2 g, 252.4 mmol) in dichloromethane (500 mL) was added to cone. sulfuric acid (96%, 350 mL) at 15-20° C. through an addition funnel under the cooling of an ice bath. The resulting mixture was stirred at room temperature for 1 hour then was poured onto ice and carefully neutralized by the addition of NH₄OH solution to pH of 9. The mixture was extracted with dichloromethane and the combined organic layers were washed with water, dried (Na₂SO₄), filtered, and concentrated in vacuo to afford the desired compound 53 (55.0 g, 80% yield) as a yellow foam with [α]_{D}=−140° (c=1.0, CHCl₃).

### Step 4. (S)-2-Amino-3,3-di(methyl-d₃)-4,4,4-d₃-butanamide (54)

A mixture of compound 53 (77.0 g, 282.7 mmol), 10% Pd/C (approximately 50% water, 20 g) and acetic acid (50 mL) in ethanol (1.2 L) was subjected to hydrogenation at 30 psi, room temperature for several days until reaction was complete. The resulting mixture was filtered through Celite and the solid washed with EtOAc. The filtrate was concentrated in vacuo, and the residue was diluted with water (1 L) then basified with 1M NaOH solution to pH of 9. The mixture was extracted with dichloromethane and the aqueous layer was concentrated in vacuo to half volume, saturated with solid NaCl, and extracted with THF. The combined extracts were dried (Na₂SO₄), filtered, and concentrated in vacuo. The residue was rinsed with toluene to remove remaining water, followed by trituration with dichloromethane to afford the desired compound 54 (38.0 g, 96% yield) as a white solid.

### Step 5. (S)-2-Amino-3,3-di(methyl-d₃)-4,4,4-d₃-butanamide hydrochloride (55)

A mixture of compound 54 (31.0 g, 222.6 mmol) in 6M aqueous HCl solution (1.5 L) was heated at reflux for 24 hours. The resulting mixture was concentrated in vacuo, leaving a solid, which was redissolved in water (500 mL) and washed with EtOAc (2×200 mL) to remove impurities from previous steps. The aqueous layer was then concentrated in vacuo, rinsed with toluene, and dried under vacuum at 50° C. to afford the HCl salt 55 (33.6 g, 85% yield) as a white solid.

### Step 6. (S)-2-(Tert-butoxycarbonylamino)-3,3-di(methyl-d₃)-4,4,4-d₃-butanoic acid (56)

To a solution of compound 55 (1.00 g, 5.66 mmol) in a mixture of dioxane (10 mL) and water (10 mL) was added triethylamine (3.16 mL, 22.6 mmol) followed by di-tert-butyl dicarbonate (1.48 g, 6.79 mmol). The resulting mixture was stirred at room temperature for 6 hours and then was extracted with heptane (2×20 mL). The aqueous fraction was cooled with an ice bath, the pH was adjusted to 2 with 1M HCl, and then the fraction was extracted with ethyl acetate (3×50 mL). The combined extracts were dried (Na₂SO₄), filtered, and concentrated in vacuo to afford 56 (1.10 g, 81% yield) as a yellow oil.

### Part b. Synthesis of (1R,2S,5S)-3-((S)-2-(3-(tert-Butyl-d₉)ureido)-3,3-di(methyl-d₃)-4,4,4-d₃-butanoyl)-6,6-di(methyl-d₃)-3-azabicyclo[3.1.0]hexane-2-carboxylic acid (83a)

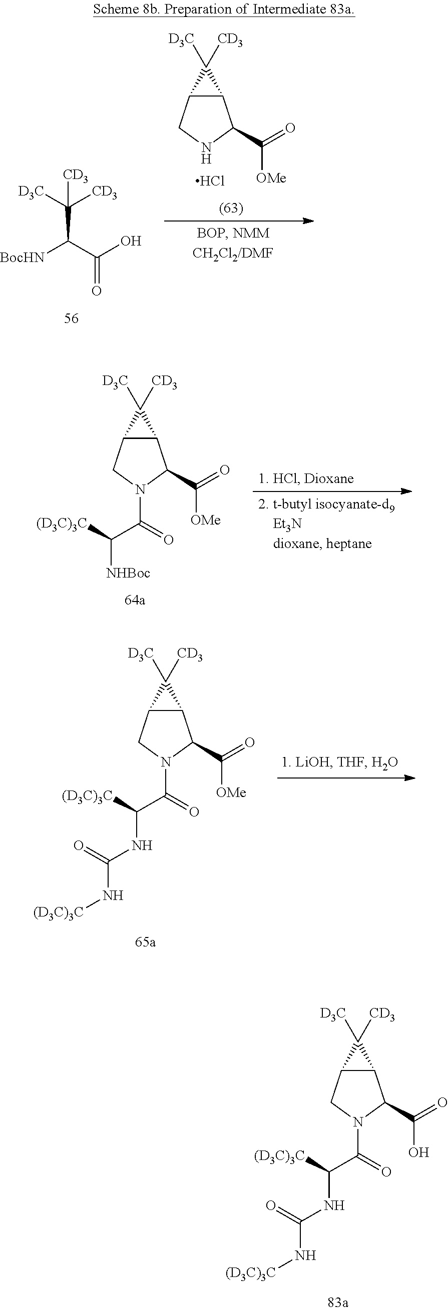

### Step 1. (1R,2S,5S)-Methyl 3-((S)-2-(tert-butoxycarbonylamino)-3,3-di(methyl-d₃)-4,4,4-d₃-butanoyl)-6,6-di(methyl-d₃)-3-azabicyclo[3.1.0]hexane-2-carboxylate (64a)

To a solution of 56 (121 mg, 0.506 mmol, see Part a for preparation) and amine hydrochloride salt 63 (128 mg, 0.607 mmol, see Example 4 for preparation) in CH₂Cl₂/DMF (3 mL, 1:1) at 0° C. was added N-methyl morpholine (167 μL, 1.52 mmol) and BOP reagent (269 mg, 0.607 mmol). The reaction was stirred at room temperature for 15 hours, diluted with 1M HCl, and extracted with CH₂Cl₂ (3×30 mL). The combined organic layers were washed with 1M HCl, saturated NaHCO₃ and brine, dried (MgSO₄), filtered, and concentrated in vacuo. The resulting residue was purified by column chromatography (SiO₂, 0-20% acetone/heptane) to afford 64a (112 mg, 56% yield). MS (M+Na): 420.4; (M+H): 398.3.

### Step 2. (1R,2S,5S)-Methyl 3-((S)-2-(3-(tert-butyl-d₉)ureido)-3,3-di(methyl-d₃)-4,4,4-d₃-butanoyl)-6,6-di(methyl-d₃)-3-azabicyclo[3.1.0]hexane-2-carboxylate (65a)

A solution of 64a (112 mg, 0.282 mmol) in 4M HCl in dioxane (5 mL) was stirred at room temperature for 3 hours then concentrated in vacuo. The resultant amine hydrochloride salt was dissolved in dioxane (300 μL) and triethylamine (197 mL, 1.40 mmol) was added with stirring. The mixture was cooled to −78° C., tert-butyl isocyanate-d₉ (130 mg, 1.20 mmol, in 10 mL heptane/dioxane 1:1, see Example 3, step 2) was added and the reaction mixture was stirred at room temperature for 15 hours. The resulting mixture was concentrated in vacuo, diluted with 1 M HCl, and extracted with CH₂Cl₂ (3×30 mL). The combined organic layers were washed with 1M HCl, saturated NaHCO₃ and brine, dried (MgSO₄), filtered, and concentrated in vacuo. The resulting residue was purified by column chromatography (SiO₂, 0-20% acetone/heptane) to afford 65a (74 mg, 65%) as a dry white foam. MS (M+Na): 428.5.

### Step 3. (1R,2S,5S)-3-((S)-2-(3-(tert-butyl-d₉)ureido)-3,3-di(methyl-d₃) 4,4,4-d₃-butanoyl)-6,6-di(methyl-d₃)-3-azabicyclo[3.1.0]hexane-2-carboxylic acid (83a)

To a solution of 65a (74 mg, 0.182 mmol) in a mixture of THF/H₂O (2 mL, 1:1) was added lithium hydroxide (7.00 mg, 0.274 mmol). The reaction was stirred for 3 hours, quenched with 1M HCl and concentrated under reduced pressure to remove THF. The resulting aqueous solution was extracted with EtOAc (3×10 mL) and the combined organic layers were dried (Na₂SO₄), filtered and concentrated in vacuo to afford 83a.

### Part c. Synthesis of (1R,2S,5S)—N-(4-Amino-1-(cyclobutyl-d₇)-1,1-d₂-3,4-dioxobutan-2-yl)-3-((S)-2-(3-(tert-butyl-d₉)ureido)-3,3-di(methyl-d₃)-4,4,4-d₃-butanoyl)-6,6-di(methyl-d₃)-3-azabicyclo[3.1.0]hexane-2-carboxamide (Compound 158).

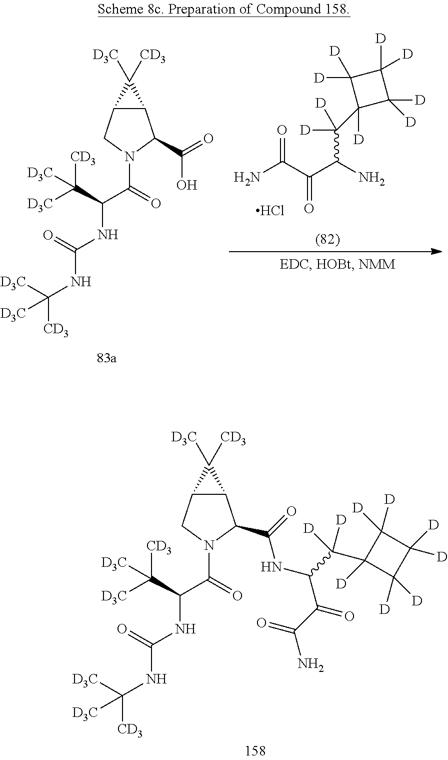

### (1R,2S,5S)—N-(4-Amino-1-(cyclobutyl-d₇)-1,1-d₂-3,4-dioxobutan-2-yl)-3-((S)-2-(3-(tert-butyl-d₉)ureido)-3,3-di(methyl-d₃)-4,4,4-d₃-butanoyl)-6,6-di(methyl-d₃)-3-azabicyclo[3.1.0]hexane-2-carboxamide (Compound 158)

To a solution of carboxylic acid 83a (55.0 mg, 0.139 mmol, prepared as described in Part b) and amine hydrochloride 82 (45.0 mg, 0.209 mmol, Example 1) in 1:1 dichloromethane/DMF (3.0 mL) at 0° C. was added EDC (53.0 mg, 0.278 mmol), HOBt (10.0 mg, 0.0700 mmol) and N-methyl morpholine (31.0 μL, 0.278 mmol). The reaction mixture was stirred at room temperature for 15 hours, concentrated in vacuo, diluted with 1M HCl, and extracted with EtOAc (3×20 mL). The combined organic layers were washed with 1M HCl, saturated NaHCO₃ and brine, dried (MgSO₄), filtered and concentrated in vacuo. The resulting residue was purified by column chromatography (SiO₂, 10-40% acetone/heptane) to afford Compound 158 with a dr=1.5:1 (S:R at chiral center 5) (28 mg, 36%) as a white solid. ¹H NMR (CDOD₃, 400 MHz): δ 7.55 (br s, 0.3H), 6.07 (br s, 1H), 5.95-5.76 (m, 1H), 4.35-4.20 (m, 2H), 4.20-3.87 (m, 3H), 3.22 (s, 1H), 1.62-1.27 (m, 2H). MS (M+H): 553.5.

### Example 4

### Synthesis of (1R,2S,5S)-Methyl 6,6-di(methyl-d₃)-3-azabicyclo[3.1.0]hexane-2-carboxylate hydrochloride (63).

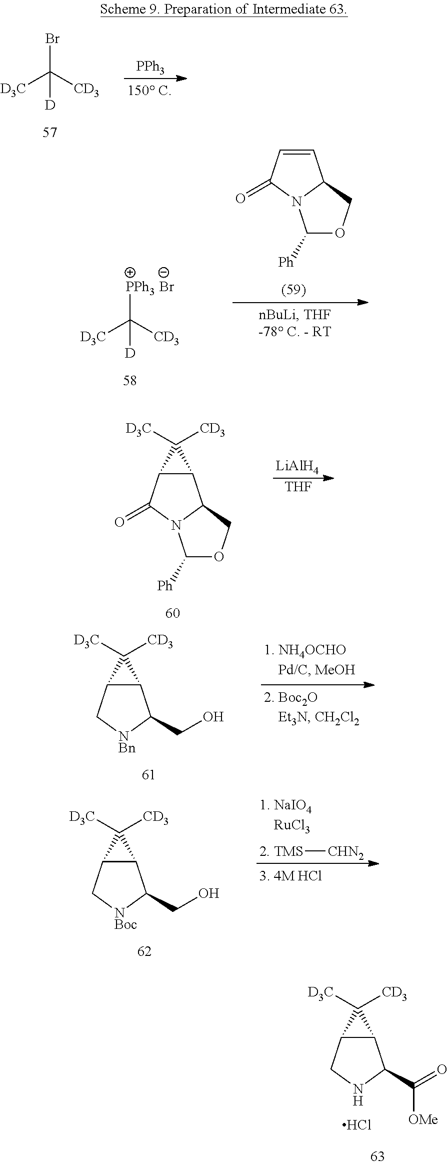

### Step 1. (Isopropyl-d₇)triphenylphosphonium bromide (58)

2-Bromopropane-d₇ 57 (3.62 mL, 38.5 mmol, Aldrich, 98% isotopic purity) and triphenyl phosphine (10.09 g, 38.5 mmol) were stirred in a sealed pressure flask at 150° C. for 15 hours. The reaction mixture was cooled to room temperature and the product crystallized from ethanol and diethyl ether. The product was filtered, washed with diethyl ether, and dried in vacuo to afford 58 as a white solid (9.95 g, 66% yield). MS (M-Br): 312.3.

### Step 2. Intermediate 60

To a solution of Wittig salt 58 (1.95 g, 4.98 mmol) in THF (15 mL) at −78° C. was added dropwise n-BuLi (2.5M in hexanes, 2.19 mL, 5.48 mmol). The reaction mixture was warmed to 0° C. and stirred for 30 minutes. The resulting red solution was cooled to −78° C. and a solution of lactam 59 (1.00 g, 4.98 mmol, commercially available from Enamine Building Blocks) in THF (10 mL) was added. The resulting mixture was stirred for 2 hours at 0° C. followed by an additional 15 hours at room temperature. The reaction was then quenched by the addition of saturated NaHCO₃ and extracted with ethyl acetate (3×50 mL). The organic layers were combined, dried (MgSO₄), filtered and concentrated in vacuo. The residue was purified via column chromatography (SiO₂, 10-30% EtOAc/heptane) to afford 60 (169 mg, 14% yield) as a pale yellow solid. MS (M+H): 250.2.

### Step 3. ((1R,2S,5S)-3-Benzyl-6,6-di(methyl-d₃)-3-azabicyclo[3.1.0]hexan-2-yl)methanol (61)

To a solution of 60 (376 mg, 1.51 mmol) in THF (3 mL) at 0° C. was added LiAlH₄ (2M in THF, 1.51 mL, 3.02 mmol). The reaction was heated to reflux for 3 hours then cooled to 0° C. and quenched by dropwise addition of 10% aqueous KHSO₄. The resulting slurry was diluted with ethyl acetate and filtered (washing the filter cake with ethyl acetate (2×10 mL). The filtrate was diluted with water (20 mL) and extracted with ethyl acetate (3×30 mL). The combined organic layers were washed with brine, dried (MgSO₄), and concentrated in vacuo to afford 61 (350 mg, 98% yield) which was used without further purification. MS (M+H): 238.3.

### Step 4. (1R,2S,5S)-Tert-Butyl 2-(hydroxymethyl)-6,6-di(methyl-d₃)-3-azabicyclo[3.1.0]hexane-3-carboxylate (62)

To a solution of 61 (350 mg, 1.48 mmol) in methanol (15 mL) was added ammonium formate (571 mg, 9.06 mmol) followed by 10% palladium on carbon (70 mg, 20 wt. %). The resulting mixture was heated to reflux, taking precautions to limit ammonium formate sublimation inside the condenser. After stirring at reflux for 2 hours, the mixture was cooled to room temperature and filtered through Celite. The Celite pad was washed with methanol (2×10 mL), followed by dichloromethane (2×20 mL). The resulting solution was then concentrated in vacuo to afford the desired deuterated amino alcohol. This material (approximately 1.48 mmol) was dissolved in dichloromethane (5 mL), and triethylamine (273 μL, 1.96 mmol), followed by di-tert-butyl dicarbonate (428 mg, 1.96 mmol), was added. The reaction mixture was stirred at room temperature for 15 hours then was diluted with 1M HCl (15 mL) and extracted with dichloromethane (3×30 mL). The combined organic layers were washed with brine, dried (Na₂SO₄), filtered, and then concentrated in vacuo. The resulting residue was purified by column chromatography (SiO₂, 0-20% EtOAc/heptane) to afford 62 (311 mg, 83%-2 step yield). MS (M-^{t}Bu): 192.3.

### Step 5. (1R,2S,5S)-Methyl 6,6-di(methyl-d₃)-3-azabicyclo[3.1.0]hexane-2-carboxylate hydrochloride (63)

To a stirred solution of 62 (311 mg, 1.26 mmol) in ethyl acetate (10 mL) and acetonitrile (10 mL) was added a solution of ruthenium trichloride monohydrate (5.50 mg, 0.0252 mmol) and sodium periodate (2.16 g, 10.0 mmol) in water (15 mL). The mixture was stirred at room temperature for 1 hour, then was filtered through Celite. The Celite pad was washed with ethyl acetate (3×5 mL) and the resulting solution was concentrated in vacuo. The resulting residue was diluted with 1M HCl (10 mL) and extracted with ethyl acetate (3×20 mL). The organic layers were combined, washed with 1M HCl, dried (Na₂SO₄), filtered and concentrated in vacuo to afford the crude deuterated acid as a dark tan solid. This acid (313 mg, 1.20 mmol) was dissolved in a mixture of benzene (5.0 mL) and methanol (0.50 mL) and a 2M solution of trimethylsilyl diazomethane in hexanes (780 μL, 1.56 mmol) was added dropwise. The yellow solution was stirred at room temperature for 15 hours and was subsequently quenched by the dropwise addition of acetic acid until effervescence ceased. The reaction was then concentrated in vacuo with several repeated heptane dilutions/concentrations to remove excess acetic acid. The resulting residue was purified by column chromatography (SiO₂, 0-30% EtOAc/heptane) to afford the pure deuterated methyl ester of 63 (154 mg). To this material was added a 4M solution of HCl in dioxane (5.0 mL) and the resulting solution was stirred at room temperature for 2 hours. The reaction was then concentrated in vacuo to afford the pure hydrochloride salt 63 (128 mg, 41%-3 step yield) as a colorless solid. MS (M+H): 176.2 (free amine).

### Example 5

### Synthesis of (1R,2S,5S)—N-(4-Amino-1-cyclobutyl-3,4-dioxobutan-2-yl)-3-((S)-2-(3-tert-butylureido)-3,3-dimethylbutanoyl)-6,6-di(methyl-d₃)-3-azabicyclo[3.1.0]hexane-2-carboxamide (Compound 134).

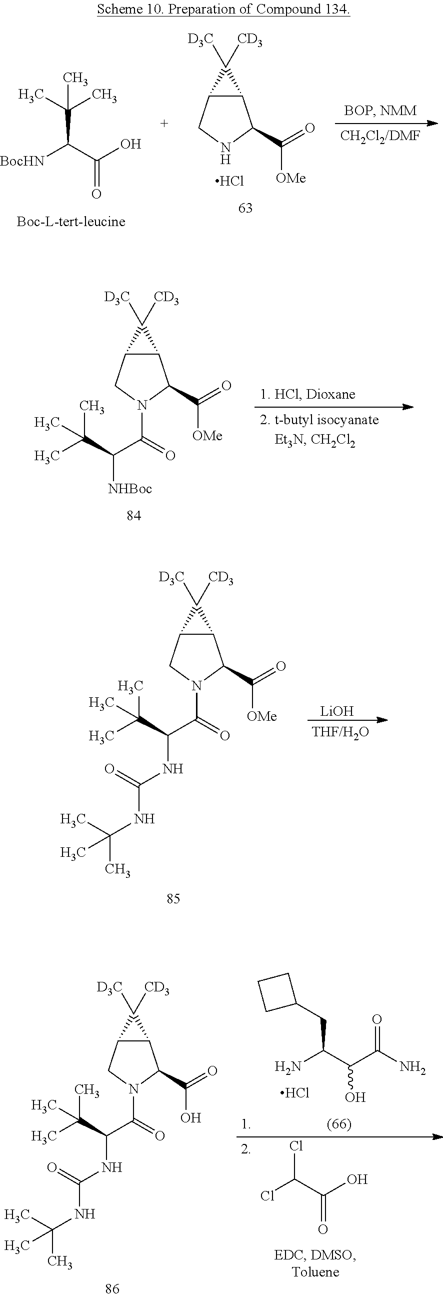

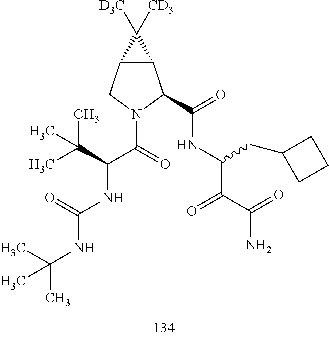

### Step 1. (1R,2S,5S)-Methyl 3-((S)-2-(tert-butoxycarbonylamino)-3,3-dimethylbutanoyl)-6,6-di(methyl-d₃)-3-azabicyclo[3.1.0]hexane-2-carboxylate (84).

To a solution of Boc-L-tert-leucine (278 mg, 1.20 mmol) and amine hydrochloride salt 63 (305 mg, 1.44 mmol, see Example 4 for preparation) in CH₂Cl₂/DMF (6 mL, 1:1) at 0° C. was added 4-methyl morpholine (396 μL, 3.60 mmol) and BOP reagent (637 mg, 1.44 mmol). The reaction was stirred at room temperature for 15 hours, diluted with 1M HCl, and extracted with CH₂Cl₂ (3×50 mL). The combined organic layers were washed successively with 1M HCl, sat. NaHCO₃ and brine, dried (MgSO₄), filtered and concentrated in vacuo. The resulting residue was purified by column chromatography (SiO₂, 0-20% acetone/heptane) to afford 84 (358 mg, 77%) as a white solid. MS (M+H): 389.4.

### Step 2. (1R,2S,5S)-Methyl 3-((S)-2-(3-tert-butylureido)-3,3-dimethylbutanoyl)-6,6-di(methyl-d₃)-3-azabicyclo[3.1.0]hexane-2-carboxylate (85).

A solution of methyl ester 84 (358 mg, 0.921 mmol) in 4M HCl in dioxane (10 mL) was stirred at room temperature for 3 hours then concentrated in vacuo. The resulting amine hydrochloride salt was directly dissolved in dichloromethane (10 mL) and triethylamine (270 μL, 1.93 mmol) was added. The mixture was cooled to −78° C. and t-butyl isocyanate (216 μL, 1.84 mmol) was added. The reaction mixture was stirred at room temperature for 15 hours, concentrated in vacuo, diluted with 1M HCl, and extracted with CH₂Cl₂ (3×50 mL). The combined organic layers were concentrated in vacuo to afford 85 as a white crunchy foam which was used without further purification. MS (M+H): 388.3.

### Step 3. (1R,2S,5S)-3-((S)-2-(3-tert-Butylureido)-3,3-dimethylbutanoyl)-6,6-di(methyl-d₃)-3-azabicyclo[3.1.0]hexane-2-carboxylic acid (86)

To a solution of 85 (357 mg, 0.921 mmol) in a mixture of THF/H₂O (10 mL, 1:1) was added lithium hydroxide (33.0 mg, 1.38 mmol). The reaction was stirred for 3 hours, then quenched with 1M HCl and concentrated in vacuo to remove THF. The resulting aqueous solution was extracted with EtOAc (3×30 mL) and the combined organic layers were dried (Na₂SO₄), filtered and concentrated to afford 86 (340 mg, 76%-4 steps). MS (M+H): 374.4.

### Step 4. (1R,2S,5S)—N-(4-Amino-1-cyclobutyl-3,4-dioxobutan-2-yl)-3-((s)-2-(3-tert-butylureido)-3,3-dimethylbutanoyl)-6,6-di(methyl-d₃)-3-azabicyclo[3.1.0]hexane-2-carboxamide (Compound 134)

To a solution of 86 (70.0 mg, 0.187 mmol) dissolved in a mixture of CH₂Cl₂/DMF (2 mL, 1:1) at 0° C. was added amine hydrochloride salt 66 (47.0 mg, 0.224 mmol, see Venkatraman, S et al, J Med Chem, 2006, 49: 6074-6086 for preparation), EDC (54.0 mg, 0.281 mmol), HOBt (38.0 mg, 0.281 mmol), and 4-methyl morpholine (82 μL, 0.748 mmol). The reaction was stirred at room temperature for 15 hours, then concentrated in vacuo. The resulting residue was diluted with 1M HCl and extracted with EtOAc (3×30 mL). The combined organic layers were washed successively with 1M HCl, sat. NaHCO₃ and brine, dried (MgSO₄), filtered and concentrated. To a solution of this material in a mixture of toluene/DMSO (4 mL, 1:1) at 0° C. was added EDC (320 mg, 1.67 mmol) and dichloroacetic acid (69.0 μL, 0.834 mmol). The reaction was stirred at room temperature for 4 hours, then diluted with sat. NaHCO₃ and extracted with CH₂Cl₂ (3×30 mL). The combined organic layers were washed successively with sat. NaHCO₃, 1M HCl and brine, dried (MgSO₄), filtered and concentrated in vacuo. The resulting residue was purified by column chromatography (SiO₂, 0-30% acetone/heptane) to afford 134 with a dr=2.5:1 (S:R at chiral center 5) (40.4 mg, 41%) as a white solid. ¹H NMR (DMSO-d₆, 400 MHz): δ 8.28 (d, J=7.3 Hz, 0.7H), 8.21 (d, J=7.8 Hz, 0.3H), 8.03 (s, 0.7H), 7.99 (s, 0.3H), 7.76 (br. s, 1H), 5.94 (s, 1H), 5.87-5.79 (m, 1H), 5.00-4.90 (m, 0.7H), 4.90-4.81 (m, 0.3H), 4.28 (s, 1H), 4.16-4.06 (m, 1H), 4.00-3.90 (m, 1H), 3.80-3.69 (m, 1H), 2.57-2.42 (m, 0.7H), 2.40-2.29 (m, 0.3H), 2.04-1.89 (m, 2H), 1.82-1.68 (m, 3H), 1.68-1.52 (m, 3H), 1.45-1.39 (m, 1H), 1.31-1.18 (m, 1H), 1.17 (br, s, 9H), 0.89 (br, s, 9H). MS (M+H): 526.5.

### Example 6

### Synthesis of (1R,2S,5S)—N-(4-Amino-1-(cyclobutyl-d₇)-1,1-d₂-3,4-dioxobutan-2-yl)-3-((S)-2-(3-tert-butylureido)-3,3-dimethylbutanoyl)-6,6-di(methyl-d₃)-3-azabicyclo[3.1.0]hexane-2-carboxamide (Compound 130).

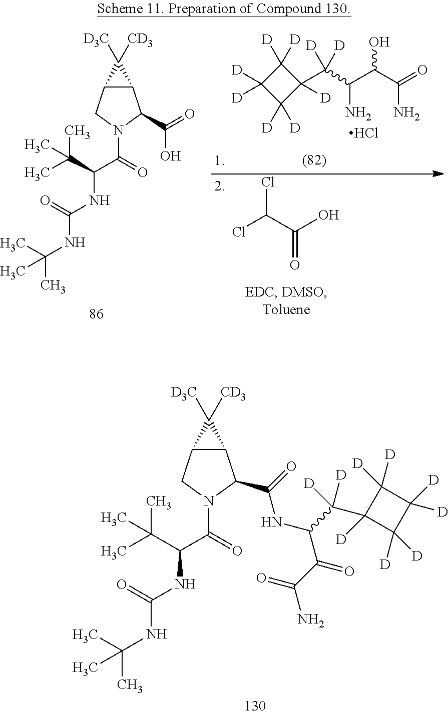

### (1R,2S,5S)—N-(4-Amino-1-(cyclobutyl-d₇)-1,1-d₂-3,4-dioxobutan-2-yl)-3-((S)-2-(3-tert-butylureido)-3,3-dimethylbutanoyl)-6,6-di(methyl-d₃)-3-azabicyclo[3.1.0]hexane-2-carboxamide (Compound 130).

To a solution of 86 (32.0 mg, 0.0850 mmol) dissolved in a mixture of CH₂Cl₂/DMF (1 mL, 1:1) at 0° C. was added amine hydrochloride salt 82 (22.0 mg, 0.102 mmol, see Example 1), EDC (25.0 mg, 0.128 mmol), HOBt (17.0 mg, 0.128 mmol), and 4-methyl morpholine (37.0 μL, 0.340 mmol). The reaction was stirred at room temperature for 15 hours, then concentrated in vacuo. The resulting residue was diluted with 1M HCl and extracted with EtOAc (3×30 mL). The combined organic layers were washed successively with 1M HCl, sat. NaHCO₃ and brine, dried (MgSO₄), filtered and concentrated. To a solution of this material in a mixture of toluene/DMSO (1.5 mL, 1:1) at 0° C. was added EDC (144 mg, 0.750 mmol) and dichloroacetic acid (31.0 μL, 0.375 mmol). The reaction was stirred at room temperature for 4 hours, then was diluted with sat. NaHCO₃ and extracted with CH₂Cl₂ (3×30 mL). The combined organic layers were washed successively with sat. NaHCO₃, 1M HCl and brine, dried (MgSO₄), filtered and concentrated in vacuo. The resulting residue was purified by column chromatography (SiO₂, 0-30% acetone/heptane) to afford 130 with a dr=1.4:1 (S:R at chiral center 5) (21.2 mg, 47%-2 steps) as a white solid. ¹H NMR (DMSO-dc, 400 MHz): δ 8.28 (d, J=7.3 Hz, 0.6H), 8.18 (d, J=7.8 Hz, 0.4H), 8.03 (s, 0.6H), 7.99 (s, 0.4H), 7.78 (br. s, 1H), 5.94 (s, 1H), 5.87-5.79 (m, 1H), 4.94 (d, J=7.3 Hz, 0.6H), 4.82 (d, J=7.3 Hz, 0.4H), 4.28 (br. s, 1H), 4.16-4.06 (m, 1H), 4.00-3.90 (m, 1H), 3.80-3.69 (m, 1H), 1.45-1.39 (m, 1H), 1.31-1.18 (m, 1H), 1.17 (br. s, 9H), 0.89 (br. s, 9H). MS (M+H): 535.5.

### Example 7

### Synthesis of 3-Amino-4-(cyclobutyl-d₇)-2-hydroxybutanamide hydrochloride (96)

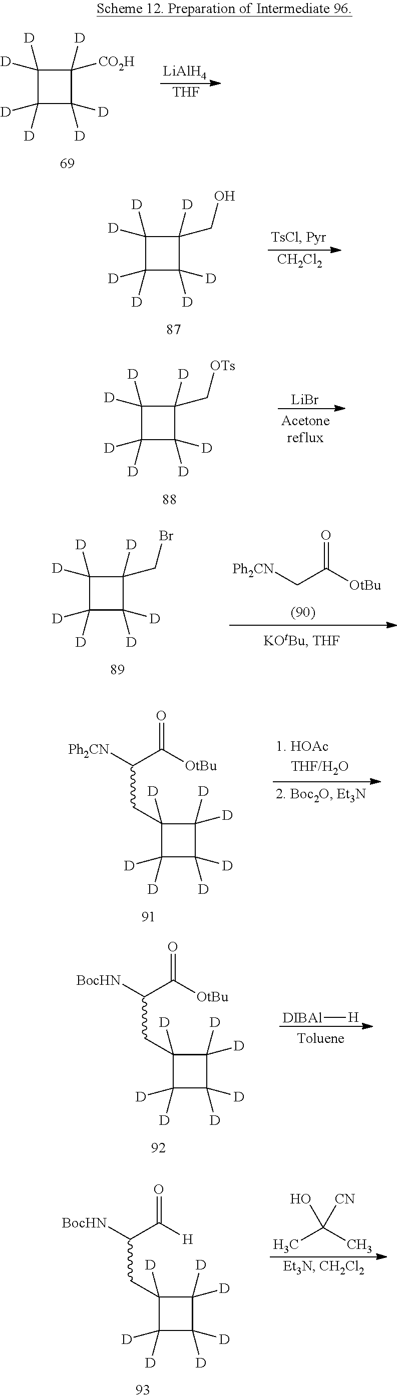

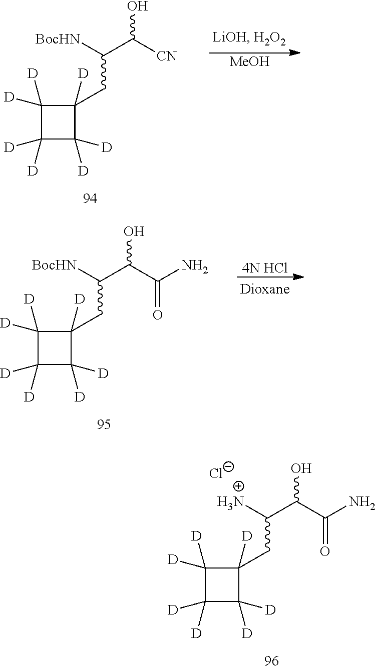

### Step 1. (Cyclobutyl-d₇)methyl 4-Methylbenzenesulfonate (88)

To a solution of 69 (1.00 g, 9.35 mmol, see Example 1) in THF (50 mL) at 0° C. was added a 2M solution of LiAlH₄ in THF (5.61 mL, 11.2 mmol). The reaction stirred at room temperature for 4 hours then was cooled to 0° C. Once at 0° C. the reaction was quenched by dropwise addition of 10% KHSO₄ until a solid white cake formed and further addition of KHSO₄ was unreactive. The cake was then broken up, filtered through Celite and washed with diethyl ether repeatedly. The resulting filtrate was concentrated in vacuo, dissolved in dichloromethane (100 mL), dried (Na₂SO₄), filtered and concentrated. The resulting alcohol (87) was dissolved in CH₂Cl₂ (10 mL), cooled to 0° C. and pyridine (2.27 mL, 2.81 mmol) was added followed by p-toluenesulfonyl chloride (1.78 g, 9.35 mmol). The reaction was stirred at room temperature for 15 hours then diluted with diethyl ether (200 mL). The resulting solution was then washed with water (100 mL), 1M HCl (3×50 mL), water (100 mL), and brine (100 mL). The organic layer was then dried (MgSO₄), filtered and concentrated to afford tosylate 88 as a clear oil (1.84 g, 80%) which was used without further purification. ¹H NMR (CDCl₃, 400 MHz): δ 7.79 (d, J=8.3 Hz, 2H), 7.34 (d, J=8.1 Hz, 2H), 3.97 (s, 2H), 2.45 (s, 3H).

### Step 2. Bromo(methyl-d₇) Cyclobutane (89)

A solution of tosylate 88 (3.21 g, 13.0 mmol) and lithium bromide (1.81 g, 20.8 mmol) in acetone (50 mL) was heated to reflux and stirred for 3 hours. The reaction was then cooled to room temperature, diluted with diethyl ether, and filtered through Celite to remove lithium tosylate. The acetone and ether were then removed via careful distillation (note: bromomethylcyclobutane bp=123° C.). The resulting residue was then diluted with ether (50 mL), washed with water (2×50 mL), dried (MgSO₄), and filtered. Diethylether was then removed via careful distillation to afford bromide 89 as a clear oil containing diethyl ether (2.02 g total, approximately 1.29 g of 89, approximately 64% by NMR). This material was carried forward into the next step.

### Step 3. tert-Butyl 3-(Cyclobutyl-d₇)-2-(diphenylmethyleneamino)propanoate (91)

To a solution of 90 (2.93 g, 9.92 mmol, purchased from Aldrich) in THF (30 mL) at −78° C. was added a 1M solution of potassium tert-butoxide in THF (9.92 mL, 9.92 mmol). The resulting solution was stirred at 0° C. for 1 hour at which time a solution of bromide 89 (1.29 g, 8.27 mmol) in THF (2 mL) was added. The reaction then stirred at room temperature for 15 hours. The THF was then removed in vacuo and the resulting residue was diluted with water (100 mL). This solution was then extracted with CH₂Cl₂ (3×50 mL), the aqueous layer was then acidified with 1M HCl and re-extracted with CH₂Cl₂ (3×50 mL). All organic layers were combined, dried (Na₂SO₄), filtered and concentrated in vacuo to afford a red oil which was purified by column chromatography (SiO₂, 0-20% EtOAc/heptane) to afford imine 91 (1.93 g, 63%). MS (M+H): 371.3.

### Step 4. tert-Butyl 2-(tert-Butoxycarbonylamino)-3-(cyclobutyl-d₇)propanoate (92)

To a solution of 91 (1.93 g, 5.21 mmol) in a mixture of THF and water (24 mL, 1:1) was added acetic acid (12 mL). The reaction mixture was stirred at room temperature for 6 hours then was quenched with sat. NaHCO₃. The resulting solution was extracted with EtOAc (3×50 mL). The organic layers were combined, dried (Na₂SO₄), filtered and concentrated in vacuo to afford a 1:1 mixture of d7-cyclobutylalanine t-butyl ester and benzophenone as observed by NMR. This mixture was then dissolved in CH₂Cl₂ (20 mL) and triethylamine (1.09 mL, 7.82 mmol) and di-tert-butyl dicarbonate (1.36 g, 6.25 mmol) were added. The reaction mixture was stirred at room temperature for 15 hours, then was diluted with 1M HCl (15 mL) and extracted with CH₂Cl₂ (3×30 mL). The combined organic layers were washed with brine, dried (Na₂SO₄), filtered and concentrated in vacuo. The resulting residue was purified by column chromatography (SiO₂, 0-20% EtOAc/heptane) to afford 92 (1.26 g, 80%) as a clear oil. ¹H NMR (CDCl₃, 400 MHz): δ 4.92 (br. d 1H), 4.08 (q, J=5.6 Hz, 1H), 1.83 (dd, J=5.3, 13.6 Hz, 1H), 1.65 (dd, J=7.6, 13.6 Hz, 1H), 1.46 (s, 9H), 1.43 (s, 9H).

### Step 5. tert-Butyl 1-(Cyclobutyl-d₇)-3-oxopropan-2-ylcarbamate (93)

To a solution of 92 (1.26 g, 4.11 mmol) in toluene (20 mL) cooled to −78° C. was added a 1M solution diisobutylaluminum hydride in CH₂Cl₂ (2.12 mL, 2.12 mmol). The reaction stirred at −78° C. for 1 hour then was quenched with methanol (20 mL). After stirring at −78° C. for an additional 15 minutes, the reaction was poured into a flask containing saturated Rochelle's salt (40 mL) and subsequently stirred at room temperature for 3.5 hours. The organic layer was separated and the remaining aqueous layer was extracted with EtOAc (3×100 mL). The combined organic layers were dried (Na₂SO₄), filtered, concentrated in vacuo and purified by column chromatography (SiO₂, 0-15% EtOAc/heptane) to afford aldehyde 93 (583 mg, 61%).

### Step 6. tert-Butyl 1-Cyano-3-(cyclobutyl-d₇)-1-hydroxypropan-2-ylcarbamate (94)

Acetone cyanohydrin (471 μL, 5.15 mmol) was added to a solution of aldehyde 93 (583 mg, 2.49 mmol) and triethylamine (420 μL, 3.01 mmol) in CH₂Cl₂ (4 mL). The reaction mixture was stirred at room temperature for 15 hours then was concentrated in vacuo. The resulting residue was diluted with 1M HCl (10 mL) and extracted with CH₂Cl₂ (3×100 mL). The organic layers were combined, washed with water then brine, dried (MgSO₄), filtered and concentrated in vacuo to afford cyanohydrin 94 (651 mg, 99%). MS (M+Na): 284.2.

### Step 7. tert-Butyl 4-Amino-1-(cyclobutyl-d₇)-3-hydroxy-4-oxobutan-2-ylcarbamate (95)

To a solution of cyanohydrin 94 (651 mg, 2.49 mmol) in methanol (10 mL) at 0° C. was added 30% H₂O₂ (1.40 mL, 13.7 mmol) followed by lithium hydroxide (72.0 mg, 2.99 mmol). The reaction mixture was stirred at 0° C. for 3 hours and was subsequently quenched at 0° C. via careful addition of excess sat. NaHSO₃. The resulting solution was diluted with water (30 mL) and extracted with CH₂Cl₂ (3×100 mL). The combined organic layers were dried (Na₂SO₄), filtered and concentrated in vacuo to afford hydroxy-amide 95 (469 mg, 67%) as a white solid. MS (M+Na): 302.3.

### Step 8. 3-Amino-4-(cyclobutyl-d₇)-2-hydroxybutanamide hydrochloride (96)

A solution of hydroxy-amide 95 (469 mg, 1.68 mmol) was stirred in 4M HCl in dioxane (5 mL) for 3 hours. The reaction was then concentrated in vacuo to afford hydroxy-amide hydrochloride salt 96 (337 mg, 93%). This material was carried forward without purification. MS (M+H): 180.2.

### Example 8

### Synthesis of (1R,2S,5S)—N-(4-Amino-1-(cyclobutyl-d₇)-3,4-dioxobutan-2-yl)-3-((S)-2-(3-tert-butylureido)-3,3-dimethylbutanoyl)-6,6-dimethyl-3-azabicyclo[3.1.0]hexane-2-carboxamide (Compound 157).

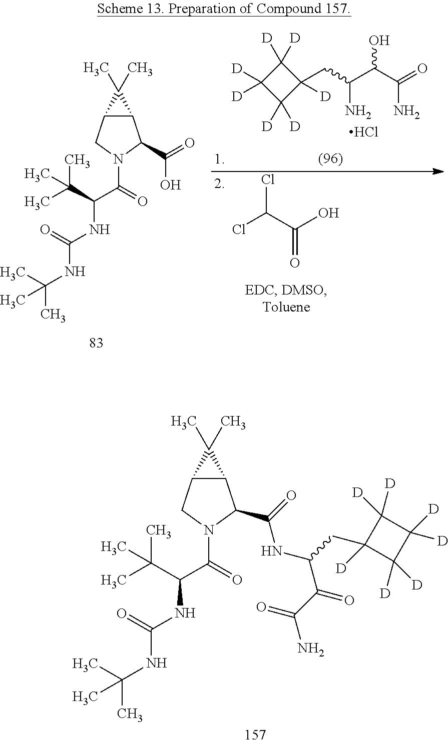

### (1R,2S,5S)—N-(4-Amino-1-(cyclobutyl-d₇)-3,4-dioxobutan-2-yl)-3-((S)-2-(3-tert-butylureido)-3,3-dimethylbutanoyl)-6,6-dimethyl-3-azabicyclo[3.1.0]hexane-2-carboxamide (Compound 157)

To a solution of 83 (71.0 mg, 0.193 mmol, see J. Med. Chem., 2006, 49: 6074-6086 for preparation) dissolved in a mixture of CH₂Cl₂/DMF (2.5 mL, 1:1) at 0° C. was added amine hydrochloride salt 96 (50.0 mg, 0.232 mmol, see Example 7), EDC (56.0 mg, 0.290 mmol), HOBt (39.0 mg, 0.290 mmol), and 4-methyl morpholine (85.0 μL, 0.772 mmol). The reaction was stirred at room temperature for 15 hours, then concentrated in vacuo. The resulting residue was diluted with 1M HCl and extracted with EtOAc (3×30 mL). The combined organic layers were washed successively with 1M HCl, sat. NaHCO₃ and brine, dried (MgSO₄), filtered and concentrated in vacuo. To a solution of this material in a mixture of toluene/DMSO (4 mL, 1:1) at 0° C. was added EDC (362 mg, 1.89 mmol) and dichloroacetic acid (78.0 μL, 0.946 mmol). The reaction stirred at room temperature for 4 hours, then was diluted with sat. NaHCO₃ and extracted with CH₂Cl₂ (3×30 mL). The combined organic layers were washed successively with sat. NaHCO₃, 1M HCl and brine, dried (MgSO₄), filtered and concentrated in vacuo. The resulting residue was purified by column chromatography (SiO₂, 0-30% acetone/heptane) to afford 157 with a dr=1.5:1 (S:R at chiral center 5) (66.0 mg, 66%) as a white solid. ¹H NMR (DMSO-d₆, 400 MHz): δ 8.28 (d, J=7.3 Hz, 0.6H), 8.18 (d, J=7.8 Hz, 0.4H), 8.03 (s, 0.6H), 7.99 (s, 0.4H), 7.77 (br. s, 1H), 5.97 (s, 1H), 5.87-5.79 (m, 1H), 4.94 (d, J=7.3 Hz, 0.6H), 4.82 (d, J=7.3 Hz, 0.4H), 4.28 (br. s, 1H), 4.16-4.06 (m, 1H), 4.00-3.90 (m, 1H), 3.80-3.69 (m, 1H), 1.78 (d, J=3.8 Hz, 0.4H), 1.74 (d, J=3.5 Hz, 0.6H), 1.65-1.50 (m, 1H), 1.46-1.39 (m, 1H), 1.27 (d, J=4.0 Hz, 0.6H), 1.25 (d, J=4.0 Hz, 0.4H), 1.17 (br. s, 9H), 1.03-0.97 (m, 3H), 0.92-0.86 (m, 9H), 0.86-0.79 (m, 3H); MS (M+H): 527.5.

### Example 9

### Synthesis of (1R,2S,5S)—N-(4-Amino-1-cyclobutyl-3,4-dioxobutan-2-yl)-6,6-dimethyl-3-((S)-4,4,4-(methyl-d₃)-2-(3-(1,1,1,3,3,3-di(methyl-d₃)-2-(methyl-d₃)propan-2-yl)ureido)-3,3-di(methyl-d₃)butanoyl)-3-azabicyclo[3.1.0]hexane-2-carboxamide (109).

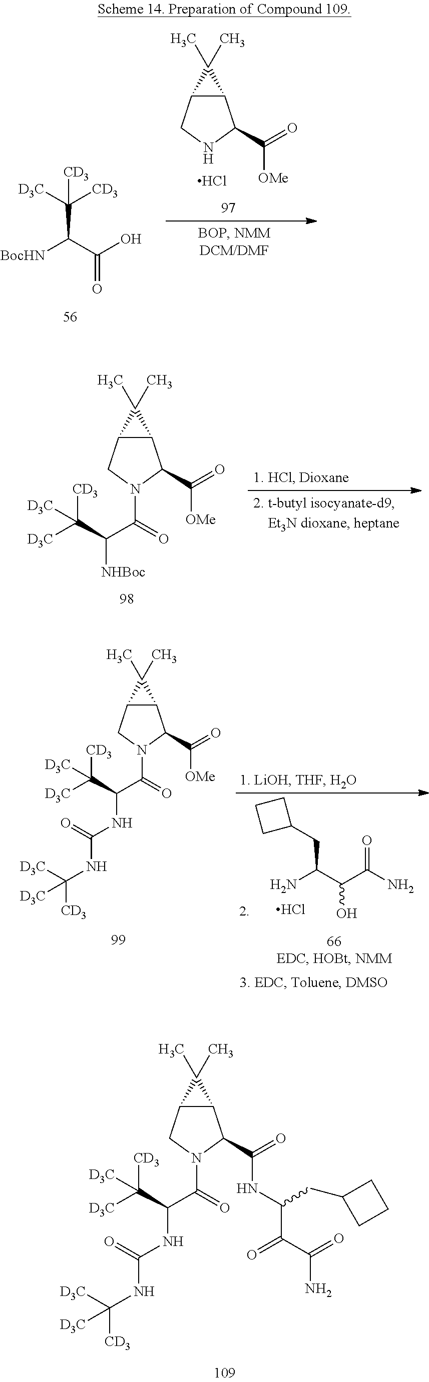

### Step 1. (1R,2S,5S)-methyl 3-((S)-2-((tert-butoxycarbonyl)amino)-4,4,4-(methyl-d₃)-3,3-di(methyl-d₃)butanoyl)-6,6-dimethyl-3-azabicyclo[3.1.0]hexane-2-carboxylate (98)

To a solution of carboxylic acid-d9 56 (258 mg, 1.07 mmol, prepared as in Example 3, Part a) and amine hydrochloride salt 97 (265 mg, 1.29 mmol; see Venkatram, S. et al. J. Med. Chem. 2006, 49, 6074-6086 for preparation) in CH₂Cl₂/DMF (6 mL, 1:1) at 0° C. was added 4-methyl morpholine (353 μL, 3.21 mmol) and BOP reagent (571 mg, 1.29 mmol). The reaction was stirred at room temperature for 15 hours, diluted with 1M HCl, and extracted with CH₂Cl₂ (3×50 mL). The combined organic layers were washed with 1M HCl, sat. NaHCO₃ and brine, dried (MgSO₄), filtered and concentrated in vacuo. The resulting residue containing methyl ester-d9 14 was used without further purification. MS (M+H): 392.4.

### Step 2. (1R,2S,5S)-methyl 6,6-dimethyl-3-((S)-4,4,4-(methyl-d₃)-2-(3-(1,1,1,3,3,3-di(methyl-d₃)-2-(methyl-d₃)propan-2-yl)ureido)-3,3-di(methyl-d₃)butanoyl)-3-azabicyclo[3.1.0]hexane-2-carboxylate (99)

A solution of methyl ester-d9 98 (˜1.29 mmol) in 4M HCl in dioxane (7 mL) was stirred at room temperature for 3 hours then concentrated in vacuo to afford the amine hydrochloride salt. This material was dissolved in dioxane (1.00 mL) and triethylamine (450 μL, 3.22 mmol) was added. The mixture was cooled to −78° C. and t-butyl isocyanate-d9 (324 mg, 3.00 mmol, in 20 mL heptane/dioxane 1:1) was added. The reaction was stirred at room temperature for 15 hours, concentrated in vacuo, diluted with 1M HCl, and extracted with CH₂Cl₂ (3×50 mL). The combined organic layers were washed with 1M HCl, sat. NaHCO₃ and brine, dried (MgSO₄), filtered and concentrated in vacuo. The resulting residue was purified by column chromatography (SiO₂, 0-20% acetone/heptane) to afford urea-d18 99 (125 mg, 30%—3 steps) as a white crunchy foam. MS (M+Na): 422.4.

### Step 3. (1R,2S,5S)—N-(4-amino-1-cyclobutyl-3,4-dioxobutan-2-yl)-6,6-dimethyl-3-((S)-4,4,4-(methyl-d₃)-2-(3-(1,1,1,3,3,3-di(methyl-d₃)-2-(methyl-d₃)propan-2-yl)ureido)-3,3-di(methyl-d₃)butanoyl)-3-azabicyclo[3.1.0]hexane-2-carboxamide (109)

To a solution of urea-d18 99 (125 mg, 0.313 mmol) in a mixture of THF/H₂O (4 mL, 1:1) was added lithium hydroxide (11.0 mg, 0.469 mmol). The reaction was stirred for 3 hours, quenched with 1M HCl and concentrated to remove THF. The resulting aqueous solution was extracted with EtOAc (3×30 mL) and the combined organic layers were dried (Na₂SO₄), filtered and concentrated. The resulting residue was dissolved in a mixture of CH₂Cl₂/DMF (4 mL, 1:1) and cooled to −20° C. To this solution was added amine hydrochloride salt 66 (86.0 mg, 0.411 mmol; see Venkatram, S. et al. J. Med. Chem. 2006, 49, 6074-6086 for preparation), EDC (98.0 mg, 0.513 mmol), HOBt (69.0 mg, 0.513 mmol), and 4-methyl morpholine (150 μL, 1.37 mmol). The reaction was stirred at −20° C. for 48 hours and concentrated in vacuo. The resulting residue was diluted with 1M HCl and extracted with EtOAc (3×30 mL). The combined organic layers were washed with 1M HCl, sat. NaHCO₃ and brine, dried (MgSO₄), filtered and concentrated to provide a light yellow foam. To a solution of this material in a mixture of toluene/DMSO (6 mL, 1:1) at 0° C. was added EDC (533 mg, 2.78 mmol) and dichloroacetic acid (115 μL, 1.39 mmol). The reaction was stirred at room temperature for 4 hours, then diluted with sat. NaHCO₃ and extracted with CH₂Cl₂ (3×30 mL). The combined organic layers were washed with sat. NaHCO₃, 1M HCl and brine, dried (MgSO₄), filtered and concentrated in vacuo. The resulting residue was purified by column chromatography (SiO₂, 0-30% acetone/heptane) to afford 109 (62.0 mg, 37%-3 steps) as a white solid and as a mixture of diastereomers. ¹H NMR (DMSO-d₆, 400 MHz) δ 8.27 (d, 0.8H, J=7.3 Hz), 8.17 (d, 0.2H, J=7.8 Hz), 8.02 (s, 0.8H), 7.97 (s, 0.2H), 7.76 (br. s, 1.0H), 7.34 (s, 0.5H), 6.90 (s, 0.4H), 5.94 (s, 0.8H), 5.93 (s, 0.2H), 5.87-5.79 (m, 1H), 5.00-4.90 (m, 0.8H), 4.90-4.81 (m, 0.2H), 4.28 (s, 0.8H), 4.27 (s, 0.2H), 4.16-4.06 (m, 1H), 4.00-3.90 (m, 1H), 3.80-3.69 (m, 1H), 2.57-2.42 (m, 0.8H), 2.40-2.29 (m, 0.2H), 2.04-1.89 (m, 2H), 1.82-1.68 (m, 3H), 1.68-1.52 (m, 3H), 1.45-1.39 (m, 1H), 1.31-1.18 (m, 1H), 1.03-0.97 (m, 3H), 0.89-0.80 (m, 3H); MS (M+H) 538.5.

### Example 10

### Synthesis of (1R,2S,5S)—N-(4-Amino-1-(cyclobutyl)-3,4-dioxobutan-2-yl)-3-((S)-2-(3-tert-butyl-4 ureido)-3,3-di(methyl-d₃)-4,4,4-d₃-butanoyl)-6,6-di(methyl-d₃)-3-azabicyclo[3.1.0]hexane-2-carboxamide (Compound 103).

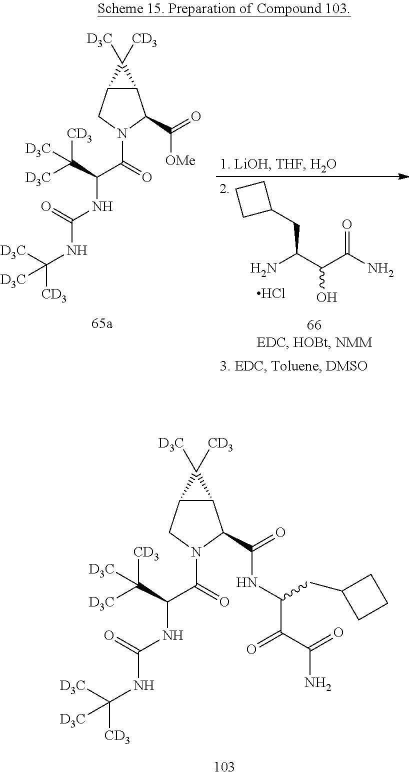

### ((1R,2S,5S)—N-(4-Amino-1-(cyclobutyl)-3,4-dioxobutan-2-yl)-3-((S)-2-(3-tert-butyl-d₉-ureido)-3,3-di(methyl-d₃)-4,4,4-d₃-butanoyl)-6,6-di(methyl-d₃)-3-azabicyclo[3.1.0]hexane-2-carboxamide (Compound 103)

To a solution of d24-urea 65a (74 mg, 0.182 mmol) in a mixture of THF/H₂O (2 mL, 1:1) was added lithium hydroxide (7.00 mg, 0.274 mmol). The reaction was stirred for 3 hours, quenched with 1M HCl and concentrated to remove THF. The resulting aqueous solution was extracted with EtOAc (3×10 mL) and the combined organic layers were dried (Na₂SO₄), filtered and concentrated. The resulting residue was dissolved in a mixture of CH₂Cl₂/DMF (2 mL, 1:1) and cooled to −20° C. To this solution was added amine hydrochloride salt 66 (46.0 mg, 0.218 mmol), EDC (52.0 mg, 0.273 mmol), HOBt (37.0 mg, 0.273 mmol), and 4-methyl morpholine (80.0 μL, 0.728 mmol). The reaction was stirred at −20° C. for 48 hours and concentrated in vacuo. The resulting residue was diluted with 1 M HCl and extracted with EtOAc (3×10 mL). The combined organic layers were washed with 1M HCl, sat. NaHCO₃ and brine, dried (MgSO₄), filtered and concentrated to provide a light yellow foam. To a solution of this material in a mixture of toluene/DMSO (3 mL, 1:1) at 0° C. was added EDC (305 mg, 1.59 mmol) and dichloroacetic acid (65.0 μL, 0.795 mmol). The reaction was stirred at room temperature for 4 hours, then diluted with sat. NaHCO₃ and extracted with CH₂Cl₂ (3×10 mL). The combined organic layers were washed with sat. NaHCO₃, 1M HCl and brine, dried (MgSO₄), filtered and concentrated in vacuo. The resulting residue was purified by column chromatography (SiO₂, 0-30% acetone/heptane) to afford 103 (20.2 mg, 23%-3 steps) as a white solid and as a mixture of diastereomers. ¹H NMR (DMSO-d₆, 400 MHz) δ 8.27 (d, 0.7H, J=7.3 Hz), 8.17 (d, 0.3H, J=7.8 Hz), 8.02 (s, 0.7H), 7.97 (s, 0.3H), 7.76 (br. s, 1.0H), 7.34 (s, 0.6H), 6.90 (s, 0.6H), 5.94 (s, 0.7H), 5.93 (s, 0.3H), 5.87-5.79 (m, 1H), 5.00-4.90 (m, 0.7H), 4.90-4.81 (m, 0.3H), 4.28 (s, 0.7H), 4.27 (s, 0.3H), 4.16-4.06 (m, 1H), 4.00-3.90 (m, 1H), 3.80-3.69 (m, 1H), 2.57-2.42 (m, 0.7H), 2.40-2.29 (m, 0.3H), 2.04-1.89 (m, 2H), 1.82-1.68 (m, 3H), 1.68-1.52 (m, 3H), 1.45-1.39 (m, 1H), 1.31-1.18 (m, 1H); MS (M+H): 436.4.

### Example 11

### Evaluation of Metabolic Stability in Human Liver Microsomes

Human liver microsomes (20 mg/mL) were obtained from Xenotech, LLC (Lenexa, Kans.). β-Nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability

7.5 mM stock solutions of Compounds 103, 109, 130, 134, 154, 157 and 158, as well as boceprevir, were prepared in DMSO. The 7.5 mM stock solutions were diluted to 12.5 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes (375 μL) were added to wells of a 96-well deep-well polypropylene plate in triplicate. 10 μL of the 12.5 μM test compound was added to the microsomes and the mixture was pre-warmed for 10 minutes. Reactions were initiated by addition of 125 μL of pre-warmed NADPH solution. The final reaction volume was 0.5 mL and contained 0.5 mg/mL human liver microsomes, 0.25 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μl of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer. 7-ethoxycoumarin (1 μM) was used as the positive control substrate.

Data Analysis

The in vitro half-lives (t_{1/2}s) for test compounds are calculated from the slopes of the linear regression of % parent remaining (ln) vs incubation time relationship using the following formula:

in vitro t_{1/2}=0.693/k, where k=−[slope of linear regression of % parent remaining(ln) vs incubation time]

Data analysis is performed using Microsoft Excel Software.

The experiment was repeated four separate times. The results of these experiments are shown in the Figure and in Tables 2a and 2b, below.

TABLE 2a
Metabolic Stability of Compounds of Formula A in HLM

| Compound | t_{1/2} (min) | % Difference^{a} |
|---|---|---|
| Boceprevir | 18.98 | — |
| 130 | 31.38 | 65.3 |
| 134 | 19.13 | 7.9 |
| 154 | 28.73 | 51.4 |
| 157 | 26.55 | 39.9 |
| 158 | 37.25 | 96.3 |

^{a}% Difference = [(deuterated species) − (nondeuterated species)](100)/(nondeuterated species)

TABLE 2b
Metabolic Stability of Alternative Boceprevir Derivatives in HLM

| Compound | t_{1/2} (min) | % Difference^{a} |
|---|---|---|
| Boceprevir | 18.98 | — |
| 103 | 25.0 | 32.0 |
| 109 | 23.2 | 21.8 |

^{a}% Difference = [(deuterated species) − (nondeuterated species)](100)/(nondeuterated species)

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of Formula A: 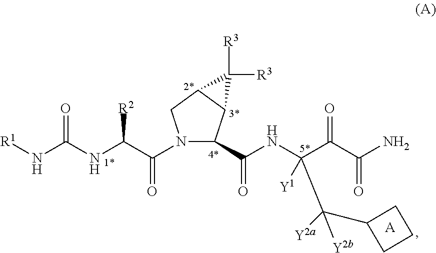
or a pharmaceutically acceptable salt thereof, wherein:
the configuration at each of chiral centers 1-4 is at least 90% of that depicted;
the configuration at chiral center 5 is from 0-100% S;
Ring A is a cyclobutyl ring having 0-7 deuterium atoms;
each of R¹ and R² is independently —C(CH₃)₃, wherein 1 to 9 hydrogen atoms are optionally replaced with deuterium atoms;
each R³ is independently selected from —CH₃, —CH₂D, —CHD₂, and —CD₃; and
each Y is independently selected from hydrogen and deuterium;
provided that at least one Y² is deuterium when R¹ and R² are simultaneously —C(CH₃)₃, R³ is —CH₃, and ring A has zero deuterium atoms; and
further provided that when R¹ and R² are —C(CD₃)₃, Y¹ is hydrogen, each Y² is hydrogen, and Ring A has zero deuterium atoms, each R³ is not CD₃ or each R³ is not CH₃.

2. The compound of claim 1, wherein R¹ is —C(CH₃)₃ or —C(CD₃)₃; R² is —C(CH₃)₃ or —C(CD₃)₃; each R³ is the same; and each Y² is the same.

3. The compound of claim 2, wherein at each R³ is —CD₃.

4. The compound of claim 1 wherein Ring A has zero or seven deuterium atoms.

5. The compound of claim 2, wherein:
each of R¹ and R² is —C(CD₃)₃;
each R³ is the same and is selected from —CD₃ and —CH₃; and
Ring A is selected from a cyclobutyl ring having 0 deuterium atoms and a cyclobutyl ring having 7 deuterium atoms.

6. The compound of claim 1 selected from any one of the compounds set forth in the table below:

| Compound | R¹ | R² | each R³ | Y¹ | each Y² | Ring A |
|---|---|---|---|---|---|---|
| 100 | C(CD₃)₃ | C(CD₃)₃ | CD₃ | D | D | D₇ |
| 101 | C(CD₃)₃ | C(CD₃)₃ | CD₃ | D | D | H₇ |
| 102 | C(CD₃)₃ | C(CD₃)₃ | CD₃ | D | H | H₇ |
| 104 | C(CD₃)₃ | C(CD₃)₃ | CD₃ | H | H | D₇ |
| 105 | C(CD₃)₃ | C(CD₃)₃ | CD₃ | D | H | D₇ |
| 106 | C(CD₃)₃ | C(CD₃)₃ | CH₃ | D | D | D₇ |
| 107 | C(CD₃)₃ | C(CD₃)₃ | CH₃ | H | D | D₇ |
| 108 | C(CD₃)₃ | C(CD₃)₃ | CH₃ | H | H | D₇ |
| 110 | C(CD₃)₃ | C(CD₃)₃ | CH₃ | H | D | H₇ |
| 111 | C(CD₃)₃ | C(CD₃)₃ | CH₃ | D | D | H₇ |
| 112 | C(CD₃)₃ | C(CD₃)₃ | CH₃ | D | H | H₇ |
| 113 | C(CD₃)₃ | C(CH₃)₃ | CD₃ | D | D | D₇ |
| 114 | C(CD₃)₃ | C(CH₃)₃ | CD₃ | H | D | D₇ |
| 115 | C(CD₃)₃ | C(CH₃)₃ | CD₃ | H | H | D₇ |
| 116 | C(CD₃)₃ | C(CH₃)₃ | CD₃ | D | D | H₇ |
| 117 | C(CD₃)₃ | C(CH₃)₃ | CD₃ | H | D | H₇ |
| 118 | C(CD₃)₃ | C(CH₃)₃ | CD₃ | D | H | H₇ |
| 119 | C(CD₃)₃ | C(CH₃)₃ | CD₃ | H | H | H₇ |
| 120 | C(CH₃)₃ | C(CD₃)₃ | CD₃ | D | D | D₇ |
| 121 | C(CH₃)₃ | C(CD₃)₃ | CD₃ | H | D | D₇ |
| 122 | C(CH₃)₃ | C(CD₃)₃ | CD₃ | H | H | D₇ |
| 123 | C(CH₃)₃ | C(CD₃)₃ | CD₃ | D | H | D₇ |
| 124 | C(CH₃)₃ | C(CD₃)₃ | CD₃ | D | D | H₇ |
| 125 | C(CH₃)₃ | C(CD₃)₃ | CD₃ | H | D | H₇ |
| 126 | C(CH₃)₃ | C(CD₃)₃ | CD₃ | H | H | H₇ |
| 127 | C(CH₃)₃ | C(CD₃)₃ | CD₃ | D | H | H₇ |
| 128 | C(CH₃)₃ | C(CH₃)₃ | CD₃ | D | D | D₇ |
| 129 | C(CH₃)₃ | C(CH₃)₃ | CD₃ | D | H | D₇ |
| 130 | C(CH₃)₃ | C(CH₃)₃ | CD₃ | H | D | D₇ |
| 131 | C(CH₃)₃ | C(CH₃)₃ | CD₃ | D | D | H₇ |
| 132 | C(CH₃)₃ | C(CH₃)₃ | CD₃ | H | D | H₇ |
| 133 | C(CH₃)₃ | C(CH₃)₃ | CD₃ | H | H | D₇ |
| 134 | C(CH₃)₃ | C(CH₃)₃ | CD₃ | H | H | H₇ |
| 135 | C(CH₃)₃ | C(CH₃)₃ | CD₃ | D | H | H₇ |
| 136 | C(CD₃)₃ | C(CH₃)₃ | CH₃ | D | D | D₇ |
| 137 | C(CD₃)₃ | C(CH₃)₃ | CH₃ | D | H | D₇ |
| 138 | C(CD₃)₃ | C(CH₃)₃ | CH₃ | H | D | D₇ |
| 139 | C(CD₃)₃ | C(CH₃)₃ | CH₃ | D | D | H₇ |
| 140 | C(CD₃)₃ | C(CH₃)₃ | CH₃ | H | D | H₇ |
| 141 | C(CD₃)₃ | C(CH₃)₃ | CH₃ | H | H | D₇ |
| 142 | C(CD₃)₃ | C(CH₃)₃ | CH₃ | D | H | H₇ |
| 143 | C(CD₃)₃ | C(CH₃)₃ | CH₃ | H | H | H₇ |
| 144 | C(CH₃)₃ | C(CD₃)₃ | CH₃ | D | D | D₇ |
| 145 | C(CH₃)₃ | C(CD₃)₃ | CH₃ | D | H | D₇ |
| 146 | C(CH₃)₃ | C(CD₃)₃ | CH₃ | H | D | D₇ |
| 147 | C(CH₃)₃ | C(CD₃)₃ | CH₃ | D | D | H₇ |
| 148 | C(CH₃)₃ | C(CD₃)₃ | CH₃ | H | D | H₇ |
| 149 | C(CH₃)₃ | C(CD₃)₃ | CH₃ | H | H | D₇ |
| 150 | C(CH₃)₃ | C(CD₃)₃ | CH₃ | D | H | H₇ |
| 151 | C(CH₃)₃ | C(CD₃)₃ | CH₃ | H | H | H₇ |
| 152 | C(CH₃)₃ | C(CH₃)₃ | CH₃ | D | D | D₇ |
| 153 | C(CH₃)₃ | C(CH₃)₃ | CH₃ | D | H | D₇ |
| 154 | C(CH₃)₃ | C(CH₃)₃ | CH₃ | H | D | D₇ |
| 155 | C(CH₃)₃ | C(CH₃)₃ | CH₃ | D | D | H₇ |
| 156 | C(CH₃)₃ | C(CH₃)₃ | CH₃ | H | D | H₇ |
| 157 | C(CH₃)₃ | C(CH₃)₃ | CH₃ | H | H | D₇ |
| 158 | C(CD₃)₃ | C(CD₃)₃ | CD₃ | H | D | D₇ |

wherein D₇ represents a cyclobutyl ring having 7 deuterium atoms; and H₇ represents a cyclobutyl ring having 0 deuterium atoms or a pharmaceutically acceptable salt of any of the foregoing.

7. The compound of claim 6 selected from: 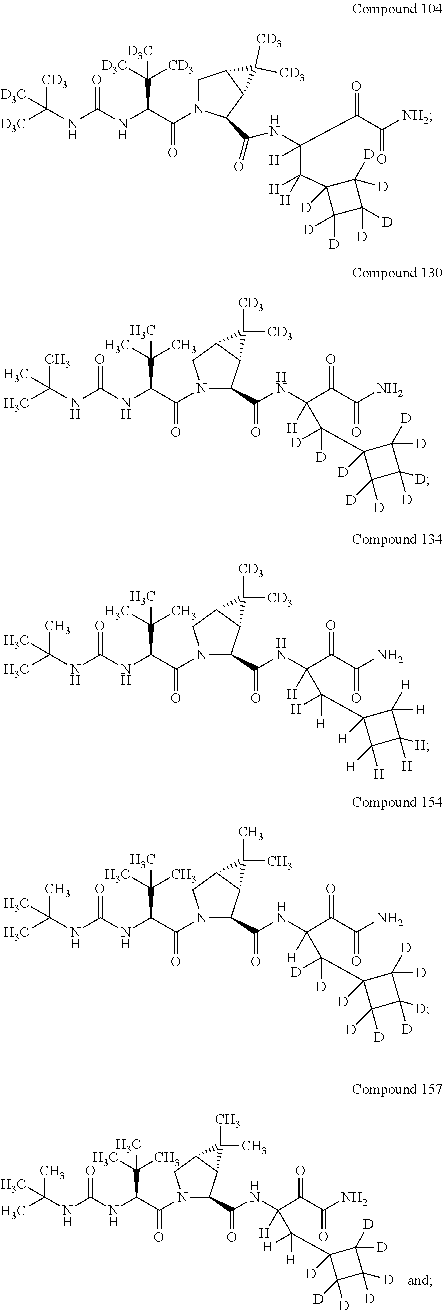 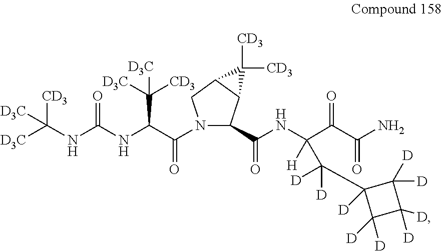
or a pharmaceutically acceptable salt of any of the foregoing.

8. The compound of claim 6, wherein any atom not designated as deuterium is present at its natural isotopic abundance.

9. The compound of claim 1, wherein the configuration at chiral center 5 is from 50-100% S.

10. The compound of claim 9, wherein the configuration at chiral center 5 is from about 60-80% S.

11. The compound of claim 1, wherein the configuration at each of the chiral centers 1-4 is at least 92% of that depicted.

12. The compound of claim 11, wherein the configuration at chiral center 5 is from 50-100% S.

13. The compound of claim 12, wherein the configuration at chiral center 5 is from about 60-80% S.

14. The compound of claim 1, wherein the configuration at each of the chiral centers 1-4 is at least 95% of that depicted.

15. The compound of claim 14, wherein the configuration at chiral center 5 is from 50-100% S.

16. The compound of claim 15, wherein the configuration at chiral center 5 is from about 60-80% S.

17. The compound of claim 1, wherein the configuration at chiral center 5 is from 0-50% S.

18. The compound of claim 17, wherein the configuration at chiral center 5 is from about 20-40% S.

19. The compound of claim 11, wherein the configuration at chiral center 5 is from 0-50% S.

20. The compound of claim 19, wherein the configuration at chiral center 5 is from about 20-40% S.

21. The compound of claim 14, wherein the configuration at chiral center 5 is from 0-50% S.

22. The compound of claim 15, wherein the configuration at chiral center 5 is from about 20-40% S.

23. A pyrogen-free pharmaceutical composition comprising a compound of claim 1 and a pharmaceutically acceptable carrier.

24. The composition of claim 23, further comprising a second therapeutic agent useful in the treatment or prevention of a hepatitis C virus (HCV) infection.

25. The composition of claim 24, wherein the second therapeutic agent is selected from PEG-interferon alpha-2a, PEG-interferon alpha-2b, ribavirin, telapravir, nitazoxanide and combinations of any two or more of the foregoing.

26. The composition of claim 25, wherein the second therapeutic agent is a combination of PEG-interferon alpha-2a and ribavirin.

27. A method of treating hepatitis C viral (HCV) infection in a subject comprising the step of administering to the subject an effective amount of a compound of claim 1 or a composition of claim 23.

28. The method of claim 27, further comprising the step of co-administering to the subject a second therapeutic agent selected from PEG-interferon alpha-2a, PEG-interferon alpha-2b, ribavirin, telapravir, nitazoxanide and combinations of any two or more of the foregoing.

29. The method of claim 28, wherein the second therapeutic agent is a combination of PEG-interferon alpha-2a and ribavirin.
